SEQUENCE LISTING (9 sequences)

SEQ ID NO: 1 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 2 - DNA, 2301 bp, Homo sapiens
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 3 - protein, 766 aa, Homo sapiens [sequence omitted]

SEQ ID NO: 4 - DNA, 5765 bp, Homo sapiens
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 5 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 6 - protein, 188 aa, Homo sapiens [sequence omitted]

SEQ ID NO: 7 - DNA, 4454 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 8 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 9 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

(19) United States
(12) Patent Application Publication
(10) Pub. No.: US 2019/0117652 A1
(43) Pub. Date: Apr. 25, 2019

(54) COMBINATION OF RAF INHIBITORS AND TAXANES

(71) Applicant: MILLENNIUM PHARMACEUTICALS, INC., Cambridge, MA (US)

(72) Inventors: Viviana BOZON, West Newton, MA (US); Katherine M. GALVIN, Newton, MA (US); Rachael L. BRAKE, Natick, MA (US); Qunli XU, Wellesley, MA (US); Karuppiah KANNAN, Newton, MA (US)

(73) Assignee: MILLENNIUM PHARMACEUTICALS, INC., Cambridge, MA (US)

(21) Appl. No.: 15/539,185

(22) PCT Filed: Dec. 22, 2015
(86) PCT No.: PCT/US15/67462

§ 371 Date: Jun. 23, 2017

Related U.S. Application Data
(60) Provisional application No. 62/096,020, filed on Dec. 23, 2014.
(60) Provisional application No. 62/242,629, filed on Oct. 16, 2015.

(51) Int. Cl. A61K 31/506; A61K 31/428; A61K 31/337; A61P 35/00

(52) U.S. Cl. CPC A61K 31/506; A61P 35/00; A61K 31/337; A61K 31/428

(57) ABSTRACT

The present disclosure relates to methods for the treatment of cancers. In particular, the disclosure provides methods for treatment of cancer by administering a Raf inhibitor in combination with a taxane. The present disclosure relates to methods of treating a subject suffering from cancer, comprising administering to the subject: (i) a Raf kinase inhibitor or a pharmaceutically acceptable salt thereof; and (ii) a taxane or a pharmaceutically acceptable salt thereof; the amount of the Raf kinase inhibitor and taxane or a pharmaceutically acceptable salt thereof is such that the combination thereof is therapeutically effective in the treatment of the cancer.

### RELATED APPLICATIONS

The present application claims priority from U.S. provisional patent application No. 62/096,064, filed Dec. 23, 2014 and U.S. provisional patent application No. 62/242,629, filed Oct. 16, 2015.

### SEQUENCE LISTING

This application contains a Sequence Listing which is submitted herewith in electronically readable format. The electronic Sequence Listing file was created on Oct. 16, 2015, is named sequencelisting.txt and has a size of 30 kb. The entire contents of the Sequence Listing in the electronic sequencelisting.txt file are incorporated herein by this reference.

This disclosure relates to methods for the treatment of cancer. In particular, the disclosure provides methods for treatment of cancer by administering Raf inhibitors in combination with a taxane.

In 2012, there were an estimated 14.1 million cancer cases around the world. This number is expected to increase to 24 million by 2035. Cancer remains the second most common cause of death in the US, accounting for nearly 1 of every 4 deaths. In 2014, there will be an estimated 1,665,540 new cancer cases diagnosed and 585,720 cancer deaths in the US. Although medical advances have improved cancer survival rates, there is a continuing need for new and more effective treatment

Cancer is characterized by uncontrolled cell reproduction. Antimitotic agents and antimicrotubule agents have been explored as targets for cancer therapy because of their important role in the cell division cycle. Inhibition of the mitotic machinery results in a diverse array of outcomes, primarily leading to cell death or arrest.

As the effect of antimitotic agents is not limited to cancer cells alone, the dose-limiting toxicities of these drugs in a clinical setting frequently manifest in rapidly dividing tissue and in the case of antimicrotubule agents are often accompanied by severe peripheral neuropathy in the case of antimicrotubule agents. Traditional antimitotic agents include those that directly interfere with microtubule dynamics, essential for mitotic spindle assembly and the subsequent alignment and segregation of DNA to daughter cells. Antimicrotubule agents, such as taxanes are currently being used in clinical setting. For example, paclitaxel and docetaxel have a similar spectrum of clinical activity including ovarian, lung, breast, bladder, and prostate cancers.

Taxanes stabilize microtubules by altering the kinetics of microtubule depolymerization. In mammalian cells grown in culture, high concentrations of paclitaxel cause the stabilization of aggregated microtubules. At lower concentrations that resemble exposures achieved in clinical settings, the primary effect of paclitaxel is to stabilize microtubules, and thereby dampen the dynamic instability of microtubules that is a requisite for efficient spindle assembly. As a result of this dampening, microtubules are unable to grow and shrink rapidly, and their ability to bind to condensed chromosomes during mitosis is compromised. Efficient chromosome alignment is thus affected, and this failure of chromosome alignment leads to mitotic delays mediated via the spindle assembly checkpoint.

It has been well established that antimitotic compounds compromise the ability of cells to execute a successful division. Cells will either fail to divide with a prolonged mitotic arrest that leads directly to cell death, or they divide abnormally, with an unequal distribution of DNA. Following such an unsuccessful division, cells may continue to cycle or undergo cell-cycle arrest or death.

The toxicities associated with paclitaxel and docetaxel are similar, and include neutropenia as the major dose limiting toxicity, along with significant peripheral neuropathy. In fact, dose reductions are frequent in heavily pretreated patients to mitigate the severity of these toxicities.

Protein kinases also play a critical role in the cell reproduction process. Specifically, the mitogen activated protein kinase (MAPK) signaling pathways consists of a kinase cascade that relays extracellular signals to the nucleus to regulate gene expression and key cellular functions. Gene expression controlled by the Ras/Raf/MEK/ERK signaling pathway regulates fundamental cellular processes including proliferation, differentiation, apoptosis, and angiogenesis. These diverse roles of Ras/Raf/MEK/ERK signaling are aberrantly activated in various types of cancer. Mutations in genes within this pathway may lead to constitutively active proteins resulting in increased cell proliferation, and resistance to apoptosis.

Raf (a serine/threonine-protein kinase) is encoded by a gene family consisting of three genes affording three Raf isoform members (B-Raf, C-Raf (Raf-1) and A-Raf). Each of these proteins share highly conserved amino-terminal regulatory regions and catalytic domains at the carboxy terminus. Although each isoform plays a role in the Ras/Raf/MEK/ERK pathway, B-Raf has been shown to be the main activator of MEK. B-Raf is recruited by Ras:GTP to the intracellular cell membrane where B-Raf becomes activated. In turn, B-Raf is responsible for activation of MEK1/2 and MEK1/2 activate ERK1/ERK2. Mutations in the B-Raf gene allow for B-Raf to signal independently of upstream signals. As a result, mutated B-Raf protein (such as V600E) causes excessive downstream signaling of MEK and ERK. This leads to excessive cell proliferation and survival and oncogenesis. Overactivation of the signaling cascade by mutated B-Raf has been implicated in multiple malignancies. B-Raf specific inhibitors (such as vemurafenib) are in fact, showing promise for the treatment of melanomas that express mutant B-Raf V600E, however the emergence of resistant disease is a growing concern.

Therefore, it would be beneficial if more effective treatment regimens could be developed. Combinations with a Raf inhibitor that inhibits more isoforms of Raf proteins than B-Raf V600E mutation could be helpful for the treatment of cancer, and might potentially even overcome the resistance to a particular anticancer agent. Given the importance of the protein kinases involved in signal transduction pathways and the cell division cycle, combinations of a Raf inhibitor with a taxane might be particularly effective. Combinations of a Raf inhibitor with a taxane may have additive, or even synergistic, therapeutic effects. Thus, there is a need for new cancer treatment regimens, including combination therapies.

### SUMMARY OF THE INVENTION

The present disclosure relates to methods of treating a subject suffering from cancer, comprising administering to the subject:
(i) a Raf kinase inhibitor or a pharmaceutically acceptable salt thereof; and
(ii) a taxane or a pharmaceutically acceptable salt thereof; the amount of the Raf kinase inhibitor and taxane or a pharmaceutically acceptable salt thereof is such that the combination thereof is therapeutically effective in the treatment of the cancer.

In some embodiments, the Raf kinase inhibitor inhibits more than the B-Raf V600 isoform of Raf proteins. In some embodiments, the Raf inhibitor is Compound A or Compound B. In some embodiments, the taxane is paclitaxel, docetaxel, or Abraxane® or a pharmaceutically acceptable salt thereof. In some embodiments, the taxane is paclitaxel or a pharmaceutically acceptable salt thereof. In some embodiments, the cancer is a K-Ras exon 2 mutation positive cancer. In some embodiments, the cancer is a B-Raf mutation positive cancer. In some embodiments, the cancer is a non-V600 B-Raf mutation positive cancer. In some embodiments, the cancer is skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, endometrial cancer, uterine cancer, liver cancer, lung cancer, prostate cancer, or colon cancer.

The present disclosure relates to methods of treating a subject suffering from cancer, comprising administering to the subject:
(i) Compound A or a pharmaceutically acceptable salt thereof; and
(ii) paclitaxel or docetaxel or a pharmaceutically acceptable salt thereof; the amount of said Compound A and paclitaxel or docetaxel or a pharmaceutically acceptable salt thereof is such that the combination thereof is therapeutically effective in the treatment of the cancer.

In some embodiments, the cancer is non-small cell lung cancer. In some embodiments, the non-small cell lung cancer is a K-Ras mutation positive cancer. In some embodiments, the cancer is a K-Ras exon 2 mutation positive cancer. In some embodiments, the non-small cell lung cancer is a B-Raf mutation positive cancer. In some embodiments, the cancer is a non-V600 B-Raf mutation positive cancer. In some embodiments, Compound A or a pharmaceutically acceptable salt thereof, is administered administered once weekly (QW) with a rest period of 6 days between each administration in an amount of up to 600 mg per dose. In some embodiments, Compound A or a pharmaceutically acceptable salt thereof, is administered once weekly in an amount of about 400 mg to about 600 mg per dose and the taxane is paclitaxel or a pharmaceutically acceptable salt thereof administered in an amount of from about 70 mg/m² to about 90 mg/m² per dose.

The present disclosure relates to a pharmaceutical composition comprising:

(i) a Raf kinase inhibitor or a pharmaceutically acceptable salt thereof; and
(ii) a taxane or a pharmaceutically acceptable salt thereof; the amount of said Raf kinase inhibitor and taxane or a pharmaceutically acceptable salt thereof is such that the combination thereof is effective for extending the duration of response.
   In one embodiment, the Raf kinase inhibitor is Compound A or Compound B.

### BRIEF DESCRIPTION OF THE DRAWINGS

FIG. 1 is a graph that shows the mean tumor volume over time in female athymic NCr-nu/nu mice bearing SK-MEL-2 human melanoma xenografts dosed with vehicle, Compound A (12.5 mg/kg, QD) or docetaxel as single agents or in combination.

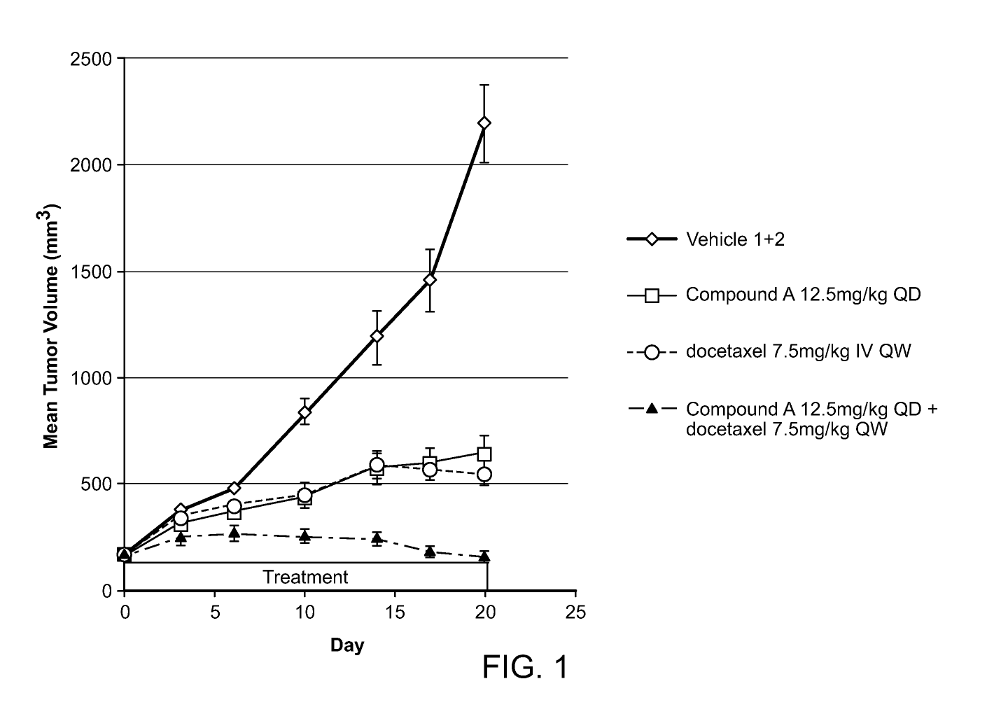

FIG. 2 a graph that shows the mean tumor volume over time in female athymic NCr-n u/nu mice bearing SK-MEL-2 human melanoma xenografts dosed with vehicle, Compound A (50 mg/kg, BIW) or docetaxel as single agents or in combination.

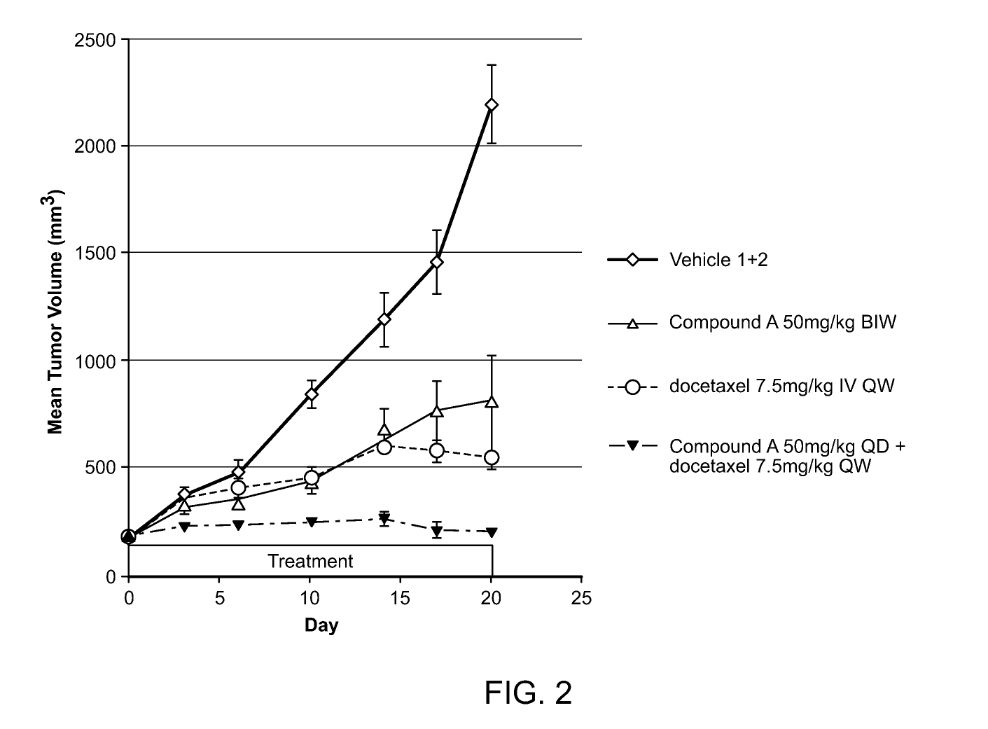

FIG. 3 is a graph that shows the average tumor volume over time in a Calu-6 lung cancer model (KRAS mutant Q^{61K}) dosed with Compound A (12.5 mg/kg, QD) or taxotere as single agents or in combination.

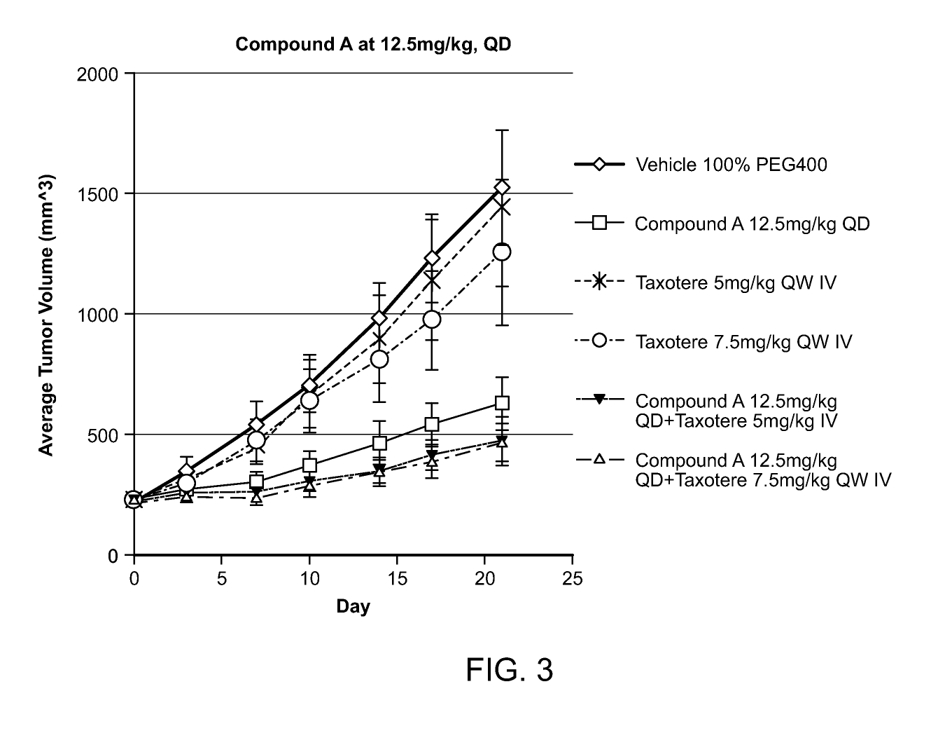

FIG. 4 is a graph that shows average tumor volume over timer in a Calu-6 lung cancer model (KRAS mutant Q^{61K}) dosed with Compound A (50 mg/kg, Q3D) or taxotere as single agents or in combination.

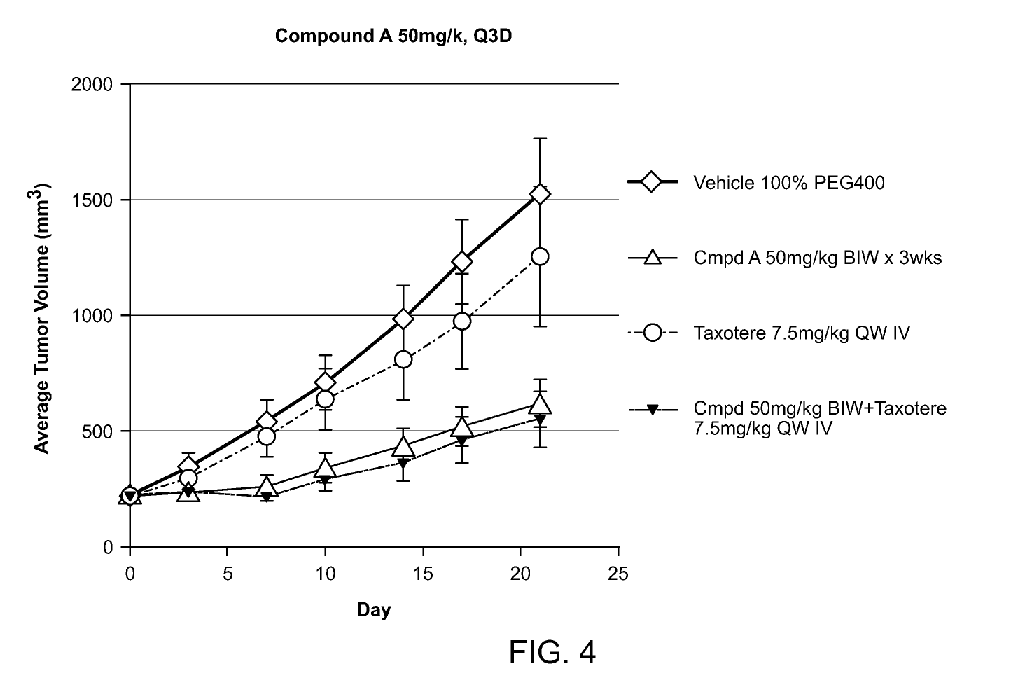

### DESCRIPTION OF THE DISCLOSURE

The present disclosure provides new combination therapies for the treatment of cancers. In particular, the present disclosure provides a method of treating a subject suffering from cancer, comprising administering to the subject: (i) a first composition comprising, as an active agent, a Raf inhibitor or a pharmaceutically acceptable salt thereof; and (ii) a second composition comprising, as an active agent, a taxane or a pharmaceutically acceptable salt thereof; the amount of said active agents being such that the combination thereof is therapeutically effective in the treatment of cancer.

Terms used herein shall be accorded the following defined meanings, unless otherwise indicated.

As used herein, the term “Raf kinase” refers to any one of a family of serine/threonine-protein kinases. The family consists of three isoform members (B-Raf, C-Raf (Raf-1), and A-Raf). Raf protein kinases are involved in the MAPK signaling pathway consisting of a kinase cascade that relays extracellular signals to the nucleus to regulate gene expression and key cellular functions. Unless otherwise indicated by context, the term “Raf kinase” is meant to refer to any Raf kinase protein from any species, including, without limitation. In one aspect, the Raf kinase is a human Raf kinase.

The term “Raf inhibitor” or “inhibitor of Raf” is used to signify a compound which is capable of interacting with one or more isoform members (B-Raf, C-Raf (Raf-1) and/or A-Raf) of the serine/threonine-protein kinase, Raf including mutant forms. Some examples of Raf mutant forms include, but are not limited to B-Raf V600E, B-Raf V600D, B-Raf V600K, B-Raf V600E+T5291 and/or B-Raf V600E+G468A.

In some embodiments, the Raf kinase is at least about 50% inhibited, at least about 75% inhibited, at least about 90% inhibited, at least about 95% inhibited, at least about 98% inhibited, or at least about 99% inhibited. In some embodiments, the concentration of Raf kinase inhibitor required to reduce Raf kinase activity by 50% is less than about 1 μM, less than about 500 nM, less than about 100 nM, less than about 50 nM, less than about 25 nM, less than about 10 nM, less than about 5 nM, or less than about 1 nM.

In some embodiments, such inhibition is selective for one or more Raf isoforms, i.e., the Raf inhibitor is selective for one or more of13-Raf (wild type), mutant B-Raf, A-Raf, and C-Raf kinase. In some embodiments, the Raf inhibitor is selective for B-Raf (wild type), B-Raf V600E, A-Raf and C-Raf. In some embodiments, the Raf inhibitor is selective for B-Raf (wild type), B-Raf V600E, A-Raf and C-Raf. In some embodiments, the Raf inhibitor is selective for B-Raf (wild type), B-Raf V600D, A-Raf and C-Raf. In some embodiments, the Raf inhibitor is selective for B-Raf (wild type), B-Raf V600K, and C-Raf. In some embodiments, the Raf inhibitor is selective for more than B-Raf V600. In some embodiments, the Raf inhibitor is selective for more than B-Raf V600E.

In some embodiments, the Raf inhibitor is selective for B-Raf and C-Raf kinases. In some embodiments, the Raf inhibitor is selective for B-Raf(wild type), B-Raf V600E and C-Raf. In some embodiments, the Raf inhibitor is selective for B-Raf (wild type), B-Raf V600D and C-Raf. In some embodiments, the Raf inhibitor is selective for B-Raf (wild type), B-Raf V600K and C-Raf. In some embodiments, the Raf inhibitor is selective for mutant B-Raf. In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

The term “pan-Raf inhibitor” is a Raf inhibitor that inhibits more than the B-Raf V600 isoform of Raf proteins.

As used herein, the term “taxane” refers to a class of diterpenes produced by the plants of the genus Taxus (yews). Examples of taxanes include, but are not limited to, paclitaxel (TAXOL®), docetaxel (TAXOTERE®), and ABRAXANE® (Paclitaxel Injection).

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to.”

As used herein, the terms “treatment,” “treat,” and “treating” are meant to include the full spectrum of intervention for the cancer from which the subject is suffering, such as administration of the combination to alleviate, slow, stop, or reverse one or more symptoms of the cancer and to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors.

The term “therapeutically effective amount” as used herein to refer to combination therapy means the amount of the combination of agents taken together so that the combined effect elicits the desired biological or medicinal response, i.e., either destroys the target cancer cells or slows or arrests the progression of the cancer in a subject. For example, the “therapeutically effective amount” as used herein to refer to combination therapy would be the amount of the Raf inhibitor and the amount of the taxane that when administered together, either sequentially or simultaneously, on the same or different days during a treatment cycle, has a combined effect that is beneficial. In some embodiments, the combined effect is additive. In some embodiments, the combined effect is synergistic. Further, it will be recognized by one skilled in the art that in the case of combination therapy with a therapeutically effective amount, as in the example above, the amount of the Raf inhibitor and/or the amount of the taxane individually may or may not be therapeutically effective.

“Cytotoxic effect,” in reference to the effect of an agent on a cell, means killing of the cell. “Cytostatic effect” means an inhibition of cell proliferation. A “cytotoxic agent” means an agent that has a cytotoxic or cytostatic effect on a cell, thereby depleting or inhibiting the growth of, respectively, cells within a cell population.

In some embodiments, the growth of cells contacted with a Raf inhibitor and a taxane is inhibited by at least about 50% as compared to growth of non-contacted cells. In some embodiments, cell proliferation of contacted cells is inhibited by at least about 75%, at least about 90%, or at least about 95% as compared to non-contacted cells. In some embodiments, the phrase “inhibiting cell proliferation” includes a reduction in the number of contacted cells, as compare to non-contacted cells. Thus, a Raf inhibitor and a taxane that inhibits cell proliferation in a contacted cell may induce the contacted cell to undergo growth retardation, to undergo growth arrest, to undergo programmed cell death (i.e., apoptosis), or to undergo necrotic cell death.

The term “subject”, as used herein, means a mammal, and “mammal” includes, but is not limited to a human. In some embodiments, the subject has been treated with an agent, e.g., a Raf inhibitor or a taxane, prior to initiation of treatment according to the method of the disclosure. In some embodiments, the subject is at risk of developing or experiencing a recurrence of a cancer.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the disclosure.

It will be apparent to one skilled in the art that certain compounds described herein may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Compounds capable of inhibiting the activity of a Raf kinase maybe be used in the methods of the instant disclosure. In some embodiments, the Raf inhibitor inhibits more isoforms of Raf kinase proteins than B-Raf V600. In some embodiment, the Raf inhibitor inhibits more isoforms of RAf kinase proteins than B-Raf V600E. In some embodiments, the Raf inhibitor inhibits B-Raf (wild-type), mutant B-Raf, A-Raf, and C-Raf. In some embodiments, the Raf inhibitor is selective for B-Raf (wild-type), B-Raf V600E, A-Raf and C-Raf. In some embodiments, the Raf inhibitor is selective for B-Raf (wild-type), B-Raf V600E, A-Raf and C-Raf. In some embodiments, the Raf inhibitor is selective for B-Raf (wild-type), B-Raf V600D, A-Raf and C-Raf. hi some embodiments, the Raf inhibitor is selective for B-Raf (wild-type), B-Raf V600K, and C-Raf. In some embodiments, the Raf inhibitor is selective for B-Raf (wild-type), B-Raf V600E and C-Raf. In some embodiments, the Raf inhibitor is selective for B-Raf (wild-type), B-Raf V600D and C-Raf. In some embodiments, the Raf inhibitor is selective for B-Raf (wild-type), B-Raf V600K and C-Raf. In some embodiments, the Raf inhibitor is selective for mutant B-Raf. In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

In particular, Raf inhibitors include the compounds described herein, as well as compounds disclosed in, for example, WO 20061065703, WO 2010/064722, WO 2011/117381, WO 2011/090738, WO 2011/161216, WO 2011/097526, WO 2011/025927, WO 2011/023773, WO 2011/147764, WO 2011/079133, and WO 2011/063159. B-Raf specific inhibitors include vemurafmib (Roche), dabrafenib (GSK), and encoratinib (Novatis). Also suitable for use in the methods of the disclosure are solvated and hydrated forms of any of these compounds. Also suitable for use in the methods of the disclosure are pharmaceutically acceptable salts of any of the compounds, and solvated and hydrated forms of such salts. These Raf inhibitors can be prepared in a number of ways well known to one skilled in the art of organic synthesis, including, but not limited to, the methods of synthesis described in detail in the above references.

hi some embodiments, the Raf inhibitor is a compound that inhibits more isoforms of Raf kinase proteins than B-Raf V600. In particular, inhibitors of Raf kinase that inhibit more isoforms of Raf kinase proteins than B-Raf V600 include for example, compounds disclosed in WO 2009/006389, and US 2013/0252977 (DP-4978/LY3009120), and including but not limited to compounds RAF-265, ARQ-736, CEP-32496, CCT 196969, CCT 241161, and REDX-04988

Raf inhibitors can be assayed in vitro or in vivo for their ability to bind to and/or inhibit Raf kinases. In vitro assays include biochemical FRET assays to measure the phophorylation of MEK by Raf kinases as a method for quantifying the ability of compounds to inhibit the enzymatic activity of Raf kinases. The compounds also can be assayed for their ability to affect cellular or physiological functions mediated by Raf kinase activity. For example in vitro assays quantitate the amount of phosphor-ERK in cancer cells. Assays for each of these activities are known in the art.

In some embodiments, the Raf inhibitor is (R)-2-(1-(6-amino-5-chloropyrimidine-4-carboxamide)ethyl)-N-(5-chloro-4-(trifluoromethyl)pyridin-2-yl)thiazole-5-carboxamide (Compound A) or a pharmaceutically acceptable salt thereof:

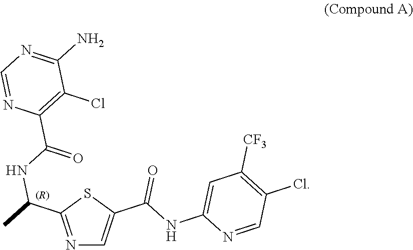

Compound A is described in WO 2009/006389.

In some embodiments, the Raf inhibitor is N-{7-cyano-6-[4-fluoro-3-({[3-(trifluoromethyl)-phenyl]acetyl}amino)phenoxy]-1,3-benzothiazol-2-yl}cyclopropanecarboxamide (Compound B) or a pharmaceutically acceptable salt thereof;

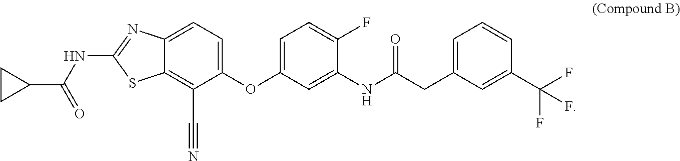 [SMILES: N#Cc1c(Oc2ccc(F)c(NC(=O)Cc3cccc(C(F)(F)F)c3)c2)ccc2nc(NC(=O)C3CC3)sc12]

Compound B is described in WO 2010/064722. Raf inhibitors, such as Compound A and Compound B that can inhibit more isoforms of Raf kinase proteins that B-Raf V600 have the ability to inhibit both Raf monomer and dimer-mediated signaling, which is a key feature that distinguishes these Raf inhibitors from recently approved B-Raf specific inhibitors (vernurafenib and dabrafenib).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt of a compound disclosed herein that, upon administration to a recipient, is capable of providing, either directly or indirectly, the compound or an active metabolite or residue thereof.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. The present disclosure also envisions the quaternization of any basic nitrogen-containing groups. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Compounds that are in the class of diterpenes produced by the plants of the genus Taxus (yews) may be used by the methods described in the instant disclosure. In particular, taxanes include but are not limited to the compounds described herein, paclitaxel (TAXOL®), docetaxel (TAXOTERE®), and ABRAXANE® (protein-bound paclitaxel for injection). In some embodiments, the taxane is paclitaxel or docetaxel or a pharmaceutically acceptable salt of paclitaxel or docetaxel. In some embodiments, the taxane is paclitaxel or a pharmaceutically acceptable salt thereof. In some embodiments, the taxane is docetaxel or a pharmaceutically acceptable salt thereof. In some embodiments, the taxane is paclitaxel. In some embodiments, the taxane is docetaxel.

In another aspect, the disclosure provides a pharmaceutical composition comprising i) a Raf inhibitor and ii) a taxane. The present disclosure provides new combination therapies for the treatment of cancers. In particular, the present disclosure provides a method of treating a subject suffering from cancer, comprising administering to the subject: (i) a first composition comprising, as an active agent, a Raf inhibitor or a pharmaceutically acceptable salt there; and (ii) a second composition comprising, as an active agent, a taxane or a pharmaceutically acceptable salt thereof; the amount of said active agents being such that the combination thereof is therapeutically effective in the treatment of cancer. In some embodiments, the Raf inhibitor is Compound A and the taxane is a paclitaxel or docetaxel. In some embodiments, the Raf inhibitor is Compound B and the taxane is paclitaxel or docetaxel.

In some embodiments, the cancer is a solid tumor. In some embodiments, the cancer is a hematological malignancy. In some embodiments, the cancer is relapsed. In one aspect, relapsed cancer is cancer which has returned after a period of time in which no cancer could be detected.

In some embodiments, the cancer is refractory. In one aspect, refractory cancer does not respond to cancer treatment; it is also known as resistant cancer. In some embodiments, the tumor is unresectable. In one aspect, an unresectable tumor is unable to be removed by surgery. In some embodiments, the cancer has not been previously treated. In some embodiments, the cancer is locally advanced. In one aspect, “locally advanced” refers to cancer that is somewhat extensive but still confined to one area. In some instances, “locally advanced” can refer to a small tumor that hasn't spread but has invaded nearby organs or tissues that make it difficult to remove with surgery alone. In some embodiments, the cancer is metastatic. In one aspect, metastatic cancer is a cancer that has spread from the part of the body where it started (the primary site) to other parts of the body.

In some embodiments, the cancer is B-Raf, N-Ras, and/or K-Ras positive cancer.

As used herein, “BRAF” or “B-Raf” refers to B-Raf proto-oncogene, serine/threonine kinase, the gene associated with the mRNA sequence assigned as GenBank Accession No. NM_004333, SEQ ID NO:1 (open reading frame is SEQ ID NO:2, nucleotides 62 to 2362 of SEQ ID NO:1), encoding GenPept Accession No. NP_004324, SEQ. ID NO:3). Other names for B-Raf include rafB1 and Noonan Syndrome 7 (NS7). B-Raf functions as a serine/threonine kinase, has a role in regulating the MAP kinase/ERKs signaling pathway and can be found on chromosome 7q.

As used herein, “KRAS” or “K-Ras” refers to v-Ki-ras2 Kirsten rat sarcoma viral oncogene homolog, the gene associated with the mRNA sequence assigned as GenBank Accession No. NM_004985, SEQ ID NO:4 (open reading frame is SEQ ID NO:5, nucleotides 193 to 759 of SEQ ID NO:4), encoding GenPept Accession No. NP_004976, SEQ ID NO:6, the predominant transcript variant of K-Ras gene on chromosome 12. Other names for K-Ras include KRAS2, and Noonan Syndrome 3 (NS3). K-Ras functions as an oncogene with GTPase activity and can be found on chromosome 12. K-Ras interacts with the cell membrane and various effector proteins, such as Akt and Cdc42, which carry out its signaling function through the cytoskeleton and effects on cell motility (Fotiadou et al. (2007) Mol. Cel. Biol. 27:6742-6755).

As used herein, “NRAS” or “N-Ras” refers to neuroblastoma RAS viral (v-ras) oncogene homolog, the gene associated with the mRNA sequence assigned as GenBank Accession No. NM_002524, SEQ ID NO:7 (open reading frame is SEQ ID NO:8, nucleotides 255 to 824 of SEQ ID NO:7), encoding GenPept Accession No. NP_002515, SEQ ID NO:9). Other names for N-Ras include Autoimmune Lymphoproliferative Syndrome type IV (ALPS4), NRAS 1, and Noonan Syndrome 6 (NS6). N-Ras functions as an oncogene with GTPase activity and can be found on chromosome 1p. N-Ras interacts with the cell membrane and various effector proteins, such as Raf and RhoA, which carry out its signaling function through the cytoskeleton and effects on cell adhesion (Fotiadou et al. (2007) Mol. Cel. Biol. 27:6742-6755).

The combination in the disclosure may be used to treat a high unmet medical need cancer. In some embodiments, the combination is used to treat a genetically defined subset of cancer. In some embodiments, the cancer is B-Raf mutation-positive cancer (i.e., the cancer has one or more B-Raf mutations). In some embodiments, the B-Raf mutation is in exon 11 or 15. In some embodiments, the B-Raf mutation is in codon 466, 469, 594, 600, or 601. In some embodiments, the B-Raf mutation is in codon 600. In some embodiments, the B-Raf mutation includes but is not limited to a V600E, V600D or V600K mutation. In some embodiments, the B-Raf mutation is V600E. In some embodiments, the B-Raf mutation is V600D. In some embodiments, the B-Raf mutation is V600K. In some embodiments, the B-Raf mutation is V600E+T5291. In some embodiments, the B-Raf mutation is V600E+G468A. “V600E mutation” means substitution of glutamic acid for valine at the amino acid position of 600. T5291 is a threonine to isoleucine B-Raf gatekeeper mutation and G468A is a B-Raf secondary mutation at G1403C in exon 11. “V600K mutation” means substitution of lysine for valine at the amino acid position of 600. “V600D mutation” means substitution of aspartic acid for valine at the amino acid position of 600. The V600K mutation results in an amino acid substitution at position 600 in B-Raf, from a valine (V) to a lysine (K) The V600K mutation results in an amino acid substitution at position 600 in B-Raf, from a valine (V) to a lysine (K)).

In some embodiments, the cancer is a non-V600 B-Raf mutation positive cancer (i.e., the cancer has one or more B-Raf mutations and the one or more mutations is not B-Raf V600). In some embodiments, the B-Raf mutation is in exon 11 or 15. In some embodiments, the B-Raf mutation is in codon 466, 469, 594, or 601. In one aspect, one or more non-V600E mutation is G466A, G466V, N581S, D594H, R146W, L613F, D565_splice, S394*, P367R, G469A, G469V, G469*, G466V, G464V, G397S, S113I, A762E, G469L, D594N, G596S, G596R, D594N, D594H, or G327_splice. In one aspect, one or more non-V600E mutations are G469R, R95T, A621_splice, V639I, Q609H, G464V, or G466V. The asterisk “*” means a stop codon.

In some embodiments, the cancer is K-Ras mutation-positive cancer (i.e., the cancer has one or more K-Ras mutations). In some embodiments, the K-Ras mutation is in exon 2. In some embodiments, the K-Ras mutation is in codon 12 or 13.

In some embodiments, the cancer is N-Ras mutation-positive cancer (i.e., the cancer has one or more N-Ras mutations). In some embodiments, the N-Ras mutation is in exon 2, 3, or 4. In some embodiments, the N-Ras mutation is in exon 2. In some embodiments, the N-Ras mutation is in exon 3. In some embodiments, the N-Ras mutation is in exon 4. In some embodiments, the N-Ras mutation is Q61R, Q61K, Q61L, Q61H, or Q611P. In some embodiments, the N-Ras mutation is Q61R mutation.

The present disclosure provides a method of treating a subject suffering from cancer. In some embodiments, the cancer is selected from skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, prostate cancer and colon cancer. In some embodiments, lung cancer includes non-small cell lung cancer (NSCLC) and small cell lung cancer (SCLC). In some embodiments, the cancer is not NSCLC. In some embodiments, the cancer is selected from skin cancer, ocular cancer, gastrointestinal cancer, thyroid cancer, breast cancer, ovarian cancer, brain cancer, laryngeal cancer, cervical cancer, lymphatic system cancer, genitourinary tract cancer, bone cancer, biliary tract cancer, endometrial cancer, uterine cancer, liver cancer, lung cancer, prostate cancer and colon cancer.

In some embodiments, the cancer is a hematological malignancy. In some embodiments, the hematological malignancy is selected from acute myelogenous leukemia (AML), chronic myelogenous leukemia (CML), chronic lymphoblastic leukemia (CLL), and myelodysplastic syndrome.

In some embodiments, the cancer is selected from thyroid cancer, ovarian cancer, melanoma, acute myelogenous leukemia (AML), and colon cancer. In some embodiments, the cancer is melanoma or colon cancer.

In some embodiments, the cancer is selected from skin cancer and gastrointestinal cancer.

In some embodiments, the cancer is skin cancer. In some embodiments, the skin cancer is melanoma. In some embodiments, the melanoma is B-Raf-mutated melanoma. In some embodiments, the melanoma is N-Ras-mutated melanoma.

In some embodiments, the cancer is lung cancer. In some embodiments, the cancer is non-small cell lung cancer (NSCLC). In some embodiments, the cancer is squamous NSCLC. In some embodiments, the cancer is non-squamous NSCLC.

In some embodiments, the cancer is gastrointestinal cancer. As used herein, “gastrointestinal cancer” includes cancer of the esophagus, stomach (also known as gastric cancer), biliary system, pancreas, small intestine, large intestine, rectum and anus). In some embodiments, the gastrointestinal cancer is adenocarcinoma of the esophagus, adenocarcinoma of the gastroesophageal junction or adenocarcinoma of the stomach. In some embodiments, the gastrointestinal cancer is stomach cancer.

In some embodiments, the cancer is colon cancer. Colon cancer is also known as colorectal (CRC), bowel, or rectum cancer.

In some embodiments, the cancer is a central nervous system cancer. In some embodiments, the central nervous system cancer is brain cancer.

In some embodiments, thyroid cancer is thyroid carcinoma.

In some embodiments, genitourinary tract cancer is bladder cancer.

In some embodiments, the hematogical malignancy is selected from acute myelogenous leukemia (AML) and chronic lymphocytic leukemia (CLL).

The Raf inhibitor and taxane are administered in such a way that they provide a beneficial effect in the treatment of a cancer. Administration can be by any suitable means provided that the administration provides the desired therapeutic effect, i.e., additivity or synergism. In some embodiments, the Raf inhibitor and taxane are administered during the same cycle of therapy, e.g., during one cycle of therapy, both the Raf kinse inhibitor and taxane are administered to the subject.

In some embodiments, the Raf inhibitor and taxane are cyclically administered to a subject. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, the treatment period during which an agent is administered is then followed by a non-treatment period of particular time duration, during which the therapeutic agents are not administered to the subject. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the subject has achieved a complete response or a partial response, at which point the treatment may be stopped. Alternatively, the period of treatment in cycling therapy may continue until the subject has achieved a complete response or a partial response, at which point the period of treatment may continue for a particular number of cycles. In some embodiments, the length of the period of treatment may be a particular number of cycles, regardless of subject response. In some other embodiments, the length of the period of treatment may continue until the subject relapses.

The amounts or suitable dosages of the Raf inhibitor depends upon a number of factors, including the nature of the severity of the condition to be treated, the particular inhibitor, the route of administration and the age, weight, general health, and response of the individual subject. In some embodiments, the suitable dose level is one that achieves inhibition of more isoforms of B-Raf kinase proteins than B-Raf V600. In some embodiments, the suitable dose level is one that achieves inhibition of B-Raf, C-Raf, A-Raf and/or B-RafV600E. In some embodiments, the suitable dose level is one that achieves inhibition of B-Raf, C-Raf, and/or B-RafV600E. In some embodiments, the suitable dose level is one that achieves a therapeutic response as measured by tumor regression, or other standard measures of disease progression, progression free survival or overall survival. In some embodiments, the suitable dose level is one that achieves this therapeutic response and also minimizes any side effects associated with the administration of the therapeutic agent.

Suitable daily dosages of inhibitors of Raf kinase can generally range, in single or divided or multiple doses, from about 10% to about 100% of the maximum tolerated dose as a single agent. In some embodiments, the suitable dosages are from about 15% to about 100% of the maximum tolerated dose as a single agent. In some some embodiments, the suitable dosages are from about 25% to about 90% of the maximum tolerated dose as a single agent. In some other embodiments, the suitable dosages are from about 30% to about 80% of the maximum tolerated dose as a single agent. In some other embodiments, the suitable dosages are from about 40% to about 75% of the maximum tolerated dose as a single agent. In some other embodiments, the suitable dosages are from about 45% to about 60% of the maximum tolerated dose as a single agent. In some embodiments, suitable dosages are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, or about 110% of the maximum tolerated dose as a single agent.

It will be understood that a suitable dosage of a Raf inhibitor may be taken at any time of the day or night. In some embodiments, a suitable dosage of a selective inhibitor of Raf inhibitor is taken in the morning. In some other embodiments, a suitable dosage of a Raf inhibitor is taken in the evening. In some other embodiments, a suitable dosage of a Raf inhibitor is taken both in the morning and the evening. It will be understood that a suitable dosage of a Raf inhibitor may be taken with or without food. In some embodiments a suitable dosage of a Raf inhibitor is taken with a meal. In some embodiments a suitable dosage of a Raf inhibitor is taken while fasting.

The present disclosure provides a method of treating a subject suffering from cancer, comprising administering to the subject: (i) a first composition comprising, as an active agent, Compound A or a pharmaceutically acceptable salt thereof; and (ii) a second composition comprising, as an active agent, paclitaxel or a pharmaceutically acceptable salt thereof; the amount of said active agents being such that the combination thereof is therapeutically effective in the treatment of cancer. In some embodiments, Compound A is administered every other day (QOD) in an amount of from about 100 mg to about 200 mg per dose. In some some embodiments, Compound A is administered QOD in an amount of about 100 mg per dose. In some embodiments, Compound A is administered QOD in an amount of about 200 mg per dose.

In some embodiments, Compound A is administered from up to about 200 mg per dose. In some embodiments, Compound A is administered from up to 200 mg per dose. Suitable QOD dosages of a Raf inhibitor e.g., Compound A can generally range, in single or divided or multiple doses, from up to about 200 mg per dose. In some embodiments, Compound A is administered as a single dose. In some embodiments, Compound A is administered as a divided dose. In some embodiments, Compound A is administered in multiple doses. Other suitable dosages of Compound A can generally range, in single or divided or multiple doses, from about 50 mg to about 200 mg per dose. Other suitable dosages of Compound A can generally range, in single or divided or multiple doses, from about 75 mg to about 200 mg per dose. In some embodiments, the suitable dosages are from about 100 mg to about 200 mg per dose. In some other embodiments, the suitable dosages are from about 150 mg to about 200 mg twice daily. In some embodiments, suitable dosages are about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg per dose. In some embodiments, the suitable dosage of Compound A is from about 100 mg to about 200 mg per dose.

Compound A is a Raf kinase inhibitor with a long half life which can support once weekly dosing (QW). In some embodiments, Compound A is administered once weekly with a rest period of 6 days between each administration. Suitable weekly dosages of a Raf inhibitor e.g., Compound A can generally range, in single or divided or multiple doses, from up to about 1500 mg once weekly (QW). Compound A can generally range, in single or divided or multiple doses, from up to 1500 mg once weekly (QW). In some embodiments, Compound A is administered from up to about 1000 mg once weekly. In some embodiments, Compound A is administered from up to 1000 mg once weekly. Once weekly means with a rest period of 6 days between each administration. In some embodiments, Compound A is administered as a single dose. In some embodiments, Compound A is administered QW in an amount of up to 600 mg per dose. In some embodiments, Compound A is administered in an amount of up to 600 mg per dose on days 2, 9, 16, and 23 of a 28-day cycle). In some embodiments, Compound A is administered as a divided dose. In some embodiments, Compound A is administered as a divided dose on the same day. In some embodiments, Compound A is administered in multiple doses. Suitable weekly dosages include from up to about 1000 mg per dose once a week with a rest period of 6 days between each administration. In some embodiments, Compound A is administered from up to 1000 mg per dose once a week with a rest period of 6 days between each administration. Other suitable weekly dosages of Compound A can generally range, in single or divided or multiple doses from about 200 mg to about 1000 mg per dose once a week. Other suitable weekly dosages of Compound A can generally range, in single or divided or multiple doses, from about 400 mg to about 1000 mg. In some embodiment, the suitable weekly dosage is from about 400 mg to about 900 mg per dose once a week. In some embodiments, the suitable weekly dosage is from about 500 mg to about 900 mg per dose once a week. In some other embodiments, the suitable weekly dosage is from about 400 mg to about 600 mg per dose once a week. In some other embodiments, the suitable weekly dosage is from about 200 mg to about 500 mg per dose once a week. In some other embodiments, the suitable weekly dosage is from about 200 mg to about 300 mg per dose once a week. In some embodiments, suitable weekly dosages are about 200 mg, 300 mg, about 400 mg, about 500 mg, about 600 mg, about 700 mg, about 800 mg, or about 900 mg per dose once a week. In some embodiments, the QW dosing schedule differentiates the combination of Compound A and taxane based on superior safety from other available therapies. In some embodiments, the QW dosing schedule differentiates the combination of Compound A and taxane based on superior efficacy from other available therapies.

The dosage of the Raf inhibitor administered to a subject will also depend on frequency of administration. In some embodiments, Compound A is administered once weekly (QW) with a rest period of 6 days between each administration. In some embodiments, Compound A is administered daily. In some embodiments, Compound A is administered every other day. In some embodiments, Compound A is administered on a 28-day cycle in which Compound A is administered on days 1, 3, 5, 8, 10, 12, 15, 17, 19, 22, 24, and 26 of a 28-day cycle. In some embodiments, Compound A is administered on a 28-day cycle in which Compound A is administered on days 2, 9, 16, and 23 of a 28-day cycle.

It will be readily apparent to those skilled in the art that other Raf inhibitor doses or frequencies of administration that provide the desired therapeutic effect are suitable for use in the present disclosure.

The taxane can be administered by any method known to one skilled in the art. For example, the taxane can be administered in the form of a composition, in one embodiment a pharmaceutical composition of a taxane and a pharmaceutically acceptable carrier, such as those described herein. In some embodiments, the pharmaceutical composition is a liquid dosage form, which can be administered via an intravenous route, such as intravenous injection or intravenous infusion. In one embodiment, paclitaxel is administered via intravenous injection. In another embodiment ABRAXANE® is administered via intravenous injection. Such pharmaceutical compositions are described in U.S. Pat. No. 6,096,331 and U.S. Pat. No. 6,506,405.

The amounts or suitable dosages of the taxane depends upon a number of factors, including the nature of the severity of the condition to be treated, the particular inhibitor, the route of administration and the age, weight, general health, and response of the individual subject. In some embodiments, the suitable dose level is one that achieves an effective exposure as measured by standard measures of effective exposure in cancer patients. In some embodiments, the suitable dose level is one that achieves a therapeutic response as measured by tumor regression, or other standard measures of disease progression, progression free survival or overall survival. In some embodiments, the suitable dose level is one that achieves this therapeutic response and also minimizes any side effects associated with the administration of the therapeutic agent.

Suitable dosages of taxanes can generally range, in single or divided or multiple doses, from about 10% to about 100% of the maximum tolerated dose as a single agent. In some embodiments, the suitable dosages are from about 15% to about 100% of the maximum tolerated dose as a single agent. In some some embodiments, the suitable dosages are from about 25% to about 90% of the maximum tolerated dose as a single agent. In some other embodiments, the suitable dosages are from about 30% to about 80% of the maximum tolerated dose as a single agent. In some other embodiments, the suitable dosages are from about 40% to about 75% of the maximum tolerated dose as a single agent. In some other embodiments, the suitable dosages are from about 45% to about 60% of the maximum tolerated dose as a single agent. In some embodiments, suitable dosages are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, or about 110% of the maximum tolerated dose as a single agent.

It will be understood that a suitable dosage of a taxane may be taken at any time of the day or night. In some embodiments, a suitable dosage of a taxane is taken in the morning. In some other embodiments, a suitable dosage of a taxane is taken in the evening. In some other embodiments, a suitable dosage of a taxane is taken both in the morning and the evening. It will be understood that a suitable dosage of a taxane may be taken with or without food. In some embodiments a suitable dosage of a taxane is taken with a meal. In some embodiments a suitable dosage of a taxane is taken while fasting.

Suitable dosages of paclitaxel can generally range, in single or divided or multiple doses, from about 40 mg/m² to about 90 mg/m² per week. Other suitable weekly dosages of paclitaxel can generally range, in single or divided or multiple doses, from about 70 mg/m² to about 90 mg/m² per week. Other suitable weekly dosages of paclitaxel can generally range, in single or divided or multiple doses, from about 60 mg/m² to about 90 mg/m² per week, or from about 60 mg/m² to about 80 mg/m² per week. In other embodiments, suitable dosages are about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², or about 90 mg/m² per week. In some embodiments, the dosage of paclitaxel is about 80 mg/m².

Additionally, it will be appreciated that the taxane can be administered as an about 3-hour, about 2-hour, about 1-hour, about 45-minute, or about 30-min IV infusion. In some embodiments, the taxane can be administered by a 1-hour IV infusion.

In some embodiments, dosage of paclitaxel is about 80 mg/m² by a 1-hour IV infusion. In some embodiments,

Suitable dosages of ABRAXANE® can generally range from about 100 mg/m² to about 260 mg/m². In some embodiments, ABRAXANE is administered about 100 mg/m² intravenously per dose. In some embodiments, ABRAXANE is administered about 100 mg/m² intravenously over 30 min per dose on days 1, 8, and 15 of a 21-day cycle. In some embodiments, ABRAXANE is administered about 100 mg/m² intravenously over 30 min per dose on days 1, 8, and 15 of a 28-day cycle.

Suitable dosages of docetaxel can generally range from about 60 mg/m² to about 100 mg/m² intravenously. In some embodiments, the dosage of docetaxel is administered about 75 mg/m² intravenously. In some embodiments, dosage of docetaxel is administered about 75 mg/m² over 60 min every 3 weeks.

In some embodiments, a first treatment period in which a first amount of the taxane is administered can be followed by another treatment period in which a same or different amount of the same or a different taxane is administered. The second treatment period can be followed by other treatment periods. During the treatment and non-treatment periods, one or more additional therapeutic agents can be administered to the subject.

In some embodiments, the taxane is administered once weekly (QW) with a rest period of 6 days between each administration. In some embodiments, the taxane is administered on a 28-day cycle in which taxane is administered on days 1, 8, and 15.

Administration of the Raf inhibitor and the taxane can be on the same or different days provided that administration provides the desired therapeutic effect. In some embodiments of the present disclosure, administration of the Raf inhibitor and the taxane will be on the same days. In some embodiments, administration of the Raf inhibitor and the taxane will be on the same and/or different days, e.g., Compound A is administered on days 1, 3, 5, 8, 10, 12, 15, 17, 19, 22, 24, and 26 of a 28-day cycle and the taxane is administered on days 1, 8, and 15 of a 28-day cycle. In some embodiments, Compound A is administered on days 2, 9, 16, and 23 of a 28-day cycle and the taxane is administered on days 1, 8, and 15 of a 28-day cycle. Alternative treatment schedules are encompassed by the present disclosure as long as they produce the desired result.

The taxane may be administered with the Raf inhibitor in a single dosage form or as a separate dosage form. When administered as a separate dosage form, the Raf inhibitor may be administered prior to, at the same time as, or following administration of the taxane.

In some embodiments, administration of a beneficial amount of the therapeutic agents encompasses administering Compound A or a pharmaceutically acceptable salt in an amount of from about 100 mg to about 200 mg per dose (measured amount of free Compound A) given on days 1, 3, 5, 8, 10, 12, 15, 17, 19, 22, 24, and 26 during the treatment cycle of 28-days in combination with administering paclitaxel or a pharmaceutically acceptable salt thereof in an amount of from about 70 mg/m² to about 90 mg/m² per dose (measured as the amount of free taxane) given on days 1, 8, and 15 during the treatment cycle of 28-days. In some embodiments, a beneficial amount of the therapeutic agents provides a synergistic benefit. In some embodiments, a beneficial amount of the therapeutic agents provides an additive benefit.

In some embodiments, the method to treat a subject suffering from cancer comprises administering to said subject a therapeutically effective amount of a combination of an amount of Compound A or a pharmaceutically acceptable salt thereof and an amount of a taxane or a pharmaceutically acceptable salt thereof. These cancer subjects include but are not limited to melanoma subjects with a B-Raf mutation, melanoma subjects who failed vemurafenib or other B-Raf inhibitors, melanoma patients with N-Ras mutation B-Raf wild type, colorectal cancer subjects with B-Raf V600E mutation B-Raf wild type, ovarian cancer subjects with B-Raf V600E mutation B-Raf wild type, lung cancer subjects with B-Raf V600E mutation B-Raf wild type, AML subjects with N-Ras mutation B-Raf wild type, liver cancer subjects with N-Ras mutation B-Raf wild type, thyroid cancer subjects with B-Raf V600E or N-Ras mutation B-Raf wild type, pancreatic cancer with B-Raf wild type, bilary tract cancer subjects with B-Raf wild type. Cancer subjects include non-small cell lung cancer subjects with K-Ras exon 2 mutation or B-Raf mutation non-V600 B-Raf. In some embodiments, the cancer subject has failed standard therapies.

The disclosure provides a method for extending duration of response to treatment in subject suffering from cancer comprising administering to the subject: (i) a first composition comprising, as an active agent, a Raf inhibitor or a pharmaceutically acceptable salt thereof; and (ii) a second composition comprising, as an active agent, a taxane or a pharmaceutically acceptable salt thereof, the amount of said active agents being such that the combination thereof is effective for extending the duration of response.

The Raf inhibitor can be administered by any method known to one skilled in the art. For example, the Raf inhibitor can be administered in the form of a first composition, in some embodiments as a pharmaceutical composition of a Raf inhibitor and a pharmaceutically acceptable carrier, such as those described herein. In some embodiments, the first composition is a solid dispersion extrudate as described in PCT application PCT/2015/022792 (WO 2015/148828), filed March 26, 2015. In some embodiments, the first composition is a solid dispersion extrudate comprising a vinylpyrrolidinone-vinyl acetate copolymer and one or more pharmaceutical acceptable excipients. In some embodiments, the copolymer is copovidone e.g, Kollidon® VA64. In some embodiments, the first composition is amorphous.

The taxane can be administered by any method known to one skilled in the art. For example, the taxane can be administered in the form of a second composition, in some embodiments a pharmaceutical composition of the taxane and a pharmaceutically acceptable carrier, such as those described herein.

If a pharmaceutically acceptable salt of the Raf inhibitor or taxane is utilized in these compositions, the salt preferably is derived from an inorganic or organic acid or base. For reviews of suitable salts, see, e.g., Berge et al, J. Pharm. Sci. 66:1-19 (1977) and Remington: The Science and Practice of Pharmacy, 20th Ed, ed. A. Gennaro, Lippincott Williams & Wilkins, 2000.

Nonlimiting examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Suitable base addition salts include, without limitation, ammonium salts, alkali metal salts, such as sodium and potassium salts, alkaline earth metal salts, such as calcium and magnesium salts, salts with organic bases, such as dicyclohexylamine, N-methyl-D-glucamine, t-butylamine, ethylene diamine, ethanolamine, and choline, and salts with amino acids such as arginine, lysine, and so forth.

Also, basic nitrogen-containing groups may be quaternized with such agents as lower alkyl halides, such as methyl, ethyl, propyl, and butyl chlorides, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The term “pharmaceutically acceptable carrier” is used herein to refer to a material that is compatible with a recipient subject. In one aspect, the subject is a mammal. In one aspect, the subject is a human. In one aspect, the material is suitable for delivering an active agent to the target site without terminating the activity of the agent. The toxicity or adverse effects, if any, associated with the carrier preferably are commensurate with a reasonable risk/benefit ratio for the intended use of the active agent.

The terms “carrier”, “adjuvant”, or “vehicle” are used interchangeably herein, and include any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating pharmaceutically acceptable compositions and known techniques for the preparation thereof. Except insofar as any conventional carrier medium is incompatible with the compounds of the disclosure, such as by producing any undesirable biological effect or otherwise interacting in a deleterious manner with any other component(s) of the pharmaceutically acceptable composition, its use is contemplated to be within the scope of this disclosure. Some examples of materials which can serve as pharmaceutically acceptable carriers include, but are not limited to, ion exchangers, alumina, aluminum stearate, lecithin, serum proteins, such as human serum albumin, buffer substances such as disodium hydrogen phosphate, potassium hydrogen phosphate, sodium carbonate, sodium bicarbonate, potassium carbonate, potassium bicarbonate, magnesium hydroxide and aluminum hydroxide, glycine, sorbic acid, or potassium sorbate, partial glyceride mixtures of saturated vegetable fatty acids, water, pyrogen-free water, salts or electrolytes such as protamine sulfate, disodium hydrogen phosphate, potassium hydrogen phosphate, sodium chloride, and zinc salts, colloidal silica, magnesium trisilicate, polyvinyl pyrrolidone, polyacrylates, waxes, polyethylene-polyoxypropylene-block polymers, wool fat, sugars such as lactose, glucose, sucrose, starches such as corn starch and potato starch, cellulose and its derivatives such as sodium carboxymethyl cellulose, ethyl cellulose and cellulose acetate, powdered tragacanth; malt, gelatin, talc, excipients such as cocoa butter and suppository waxes, oils such as peanut oil, cottonseed oil, safflower oil, sesame oil, olive oil, corn oil and soybean oil, glycols such as propylene glycol and polyethylene glycol, esters such as ethyl oleate and ethyl laurate, agar, alginic acid, isotonic saline, Ringer's solution, alcohols such as ethanol, isopropyl alcohol, hexadecyl alcohol, and glycerol, cyclodextrins, lubricants such as sodium lauryl sulfate and magnesium stearate, petroleum hydrocarbons such as mineral oil and petrolatum. Coloring agents, releasing agents, coating agents, sweetening, flavoring and perfuming agents, preservatives and antioxidants can also be present in the composition, according to the judgment of the formulator.

The pharmaceutical compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired.

In some embodiments, the compositions of the disclosure are formulated for pharmaceutical administration to a mammal. In one aspect, for pharmaceutical administration to a human being. Such pharmaceutical compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intravenously, or subcutaneously. The formulations of the disclosure may be designed to be short-acting, fast-releasing, or long-acting. Still further, compounds can be administered in a local rather than systemic means, such as administration (e.g., by injection) at a tumor site.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar--agar, calcium carbonate, potato or tapioca starch, alginic acid, certain silicates, and sodium carbonate, e) solution retarding agents such as paRaffin, absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, drapes, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compositions for use in the method of the disclosure may be formulated in unit dosage form for ease of administration and uniformity of dosage. The expression “unit dosage form” as used herein refers to a physically discrete unit of agent appropriate for the subject to be treated. It will be understood, however, that the total daily usage of the compounds and compositions of the present disclosure will be decided by the attending physician within the scope of sound medical judgment. A unit dosage form for parenteral administration may be in ampoules or in multi-dose containers.

The disclosure includes a kit, comprising (i) a first composition comprising, as an active agent, a Raf inhibitor or a pharmaceutically salt thereof; and (ii) a second composition comprising, as an active agent, taxane or a pharmaceutically acceptable salt thereof; and instructions for administering the first composition in combination with the second composition.

The disclosure includes a kit, comprising (i) a first composition comprising, as an active agent, a Raf inhibitor or a pharmaceutically salt thereof; and (ii) a second composition comprising, as an active agent, taxane or a pharmaceutically acceptable salt thereof when used to treat cancer in a subject; and instructions for administering the first composition in combination with the second composition.

Vemurafenib (Roche) was approved by the United States Food and Drug Administration (FDA) for treatment of melanoma patients with B-Raf V600E mutation. More recently, dabrafenib (B-Raf inhibitor) and trametinib (MEK inhibitor) were approved for patients with B-Raf V600E positive melanoma. Both drugs significantly improved the median progression-free survival compared with chemotherapy in Phase 3 studies. As is the case with vemurafmib, however, these responses are considered short-lived (Lancet (2012; 380:358-365), N Engl J Med 2012; 367:107-114). Similar to many other targeted therapies, the acquired resistance to B-Raf inhibition presents a therapeutic challenge to long-term survival benefit in this patient population.

To improve the benefit of B-Raf inhibitors, research continues to identify the mechanisms which render mutant B-Raf expressing melanoma cells resistant to vemurafenib. Recent studies have indicated that reactivation of the MAPK pathway is a mechanism of resistance to B-Raf inhibition. Resistant mechanisms primarily involve reactivation of ERK signaling through bypass mechanisms that are either Ras/Raf dependent, such as N-Ras activation, Nazarian et al, Nature. 2010, 468: 973-7, H-Ras activation (Su et al, New England Journal of Medicine. 2012, 366: 207-215) or C-Raf upregulation, (Johannessen et al, Nature. 2010, 468: 968-72; Montagut et al, Cancer Res. 2008, 68: 4853-61), aberrantly spliced variants of B-Raf V600E (Poulikakos et al, Nature. 2011, 480: 387-390, or Ras/Raf independent (Tp12/COT overexpression) Johannessen et al, Nature. 2010, 468: 968-72. Consequently, multiple mechanisms could attenuate the effect of B-Raf inhibition on MAPK signaling in B-Raf mutant cancers. Although a gatekeeper mutation of B-Raf (T529I) that could cause resistance to B-Raf inhibition has not yet been clinically identified, such a mutation has been experimentally demonstrated to cause resistance, Whittaker et al, Sci Transl Med. 2010, 2(35): ra41. Recent studies have also suggested that activation of MAPK-redundant signaling pathways by RTKs such as IGF-IR or PDGFRβ could play a role in acquired resistance to B-Raf inhibition; Nazarian et al, Nature. 2010, 468: 973-7; Villanueva et al, Cancer Cell. 2010, 18: 683-95; Shi et al, Cancer Res. 2011, 71: 5067-74. It is clear that MAPK reactivation is involved in many of these resistance mechanisms. A pan Raf inhibitor is expected to block MAPK reactivation.

Additionally, B-Raf specific inhibitors including vemurafenib and its close analogue N-[3-(5-chloro-1H-pyrrolo[2,3-b]pyridine-3-carbonyl)-2,4-difiuorophenyl]p-ropane-1-sulfonamide (PLX4720; a commercially available selective B-Raf inhibitor) were demonstrated to induce paradoxical pathway activation through dimerization with other Raf isoforms in a B-Raf wild type background, Hatzivassiliou G, et al. Nature, 2010, 464: 431-435; Poulikakos et al, Nature, 2010, 464: 427-430; Heidorn, et al, Cell, 2010, 140: 209-221. Vemurafenib is believed to activate the Raf/MEK/ERK pathway through binding B-Raf wild type and stimulating B-Raf-C-Raf dimerization. This paradoxical pathway activation by B-Raf specific inhibition is believed to be a major reason of skin side effects (such as squamous cell carcinoma) in some melanoma patients treated with vemurafenib. Vemurafenib is not approved for treatment of cancer patients with B-Raf wild type genetic background due to its paradoxical pathway activation activity in this genetic background.

Compound A is a Raf kinase inhibitor capable of inhibiting more isoforms of Raf kinase proteins than B-Raf V600. Specifically, Compound A inhibits B-Raf, C-Raf, and B-Raf V600E mutation (see Example 1). Due to its pan-Raf activities, Compound A is active against tumor cells with MAPK pathway activation by upstream signaling such as N-Ras mutation and K-Ras mutation, both with B-Raf wild type genetic background. Therefore, Compound A has the potential for treating cancer patients with B-Raf mutation (such as melanoma, colorectal, lung (NSCLC), ovarian and thyroid carcinoma) or N-Ras mutation, B-Raf wild type (such as melanoma, AML, CML, ALL, CLL, liver cancer), (Schubbert et al, Nature Reviews Cancer, 2007, 7: 295; Pylayeva-Gupta et al, Nature Reviews Cancer, 2011, 11: 761). Compound A is also active against melanoma tumor cells which developed resistance to vemurafenib. Therefore, it is believed that the Compound A, in combination with a taxane, will be effective for melanoma patients who have failed vemurafenib or other B-Raf inhibitors. In some embodiments, the combination of Compound A with paclitaxel is a better-tolerated combination that leads to the longest duration of therapy. The present disclosure relates to a method for extending duration of response to treatment in subject suffering from cancer comprising administering to the subject a pharmaceutical composition described herein.

The present disclosure relates to a method for determining whether to treat a subject suffering from cancer with a pharmaceutical composition described herein, said method comprising:

a) measuring at least one characteristic of at least one or more B-Raf, N-Ras and/or K-Ras markers associated with gene mutation in a subject sample comprising tumor cells;

b) identifying whether the at least one characteristic measured in step a) is informative for outcome upon treatment with the pharmaceutical composition; and

c) determining to treat the subject with the pharmaceutical composition if the informative characteristic indicates that the tumor cells comprise at least one marker gene with a B-Raf, N-Ras and/or K-Ras mutational status that indicates a favorable outcome to treatment with the pharmaceutical composition.

The present disclosure relates to a method of treating a subject suffering from cancer by administering to the subject a pharmaceutical composition described herein, said method comprising:

a) measuring at least one characteristic of at least one or more B-Raf, N-Ras and/or K-Ras markers associated with gene mutation in a subject sample comprising tumor cells;

b) identifying whether the at least one characteristic measured in step a) is informative for outcome upon treatment with the pharmaceutical composition; and

c) determining to treat the subject with the pharmaceutical composition if the informative characteristic indicates that the tumor cells comprise at least one marker gene with a B-Raf, N-Ras and/or K-Ras mutational status that indicates a favorable outcome to treatment with the pharmaceutical composition.

The present disclosure relates to a method for determining an increased likelihood of pharmacological effectiveness of treatment by a pharmaceutical composition of claim 16 or 17 in a subject diagnosed with cancer, said method comprising: subjecting a nucleic acid sample from a cancer (tumor) sample from the subject to B-Raf, N-Ras or K-Ras mutational testing or PCR, wherein the presence of at least one mutation in B-Raf, N-Ras or K-Ras gene indicates an increased likelihood of pharmacological effectiveness of the treatment.

The present disclosure relates to a method for treating a subject suffering from cancer by administering to a subject a pharmaceutical composition described herein, said method comprising: subjecting a nucleic acid sample from a cancer (tumor) sample from the subject to B-Raf, N-Ras or K-Ras mutational testing or PCR, wherein the presence of at least one mutation in B-Raf, N-Ras or K-Ras gene indicates an increased likelihood of pharmacological effectiveness of the treatment.

The present disclosure relates to a method of treating a subject having cancer, said method comprising:

i) obtaining a nucleic acid sample from a cancer sample from said subject;

ii) subjecting the sample to B-Raf, N-Ras or K-Ras mutational testing or PCR and identifying the presence of at least one mutation in B-Raf, N-Ras, or K-Ras gene; and administering an effective amount of a pharmaceutical composition described herein to the subject in whose sample the presence of at least one mutation in B-Raf, N-Ras or K-Ras gene is identified

In some embodiments, a mutation in a marker can be identified by sequencing a nucleic acid, e.g., a DNA, RNA, cDNA or a protein correlated with the marker gene, e.g., a genotype marker gene, e.g., B-Raf or N-Ras. There are several sequencing methods known in the art to sequence nucleic acids. A nucleic acid primer can be designed to bind to a region comprising a potential mutation site or can be designed to complement the mutated sequence rather than the wild type sequence. Primer pairs can be designed to bracket a region comprising a potential mutation in a marker gene. A primer or primer pair can be used for sequencing one or both strands of DNA corresponding to the marker gene. A primer can be used in conjunction with a probe, e.g., a nucleic acid probe, e.g., a hybridization probe, to amplify a region of interest prior to sequencing to boost sequence amounts for detection of a mutation in a marker gene. Examples of regions which can be sequenced include an entire gene, transcripts of the gene and a fragment of the gene or the transcript, e.g., one or more of exons or untranslated regions or a portion of a marker comprising a mutation site. Examples of mutations to target for primer selection and sequence or composition analysis can be found in public databases which collect mutation information, such as Database of Genotypes and Phenotypes (dbGaP) maintained by the National Center for Biotechnology Information (Bethesda, Md.) and Catalogue of Somatic Mutations in Cancer (COSMIC) database maintained by the Wellcome Trust Sanger Institute (Cambridge, UK).

Sequencing methods are known to one skilled in the art. Examples of methods include the Sanger method, the SEQUENOM™ method and Next Generation Sequencing (NGS) methods. The Sanger method, comprising using electrophoresis, e.g., capillary electrophoresis to separate primer-elongated labeled DNA fragments, can be automated for high-throughput applications. The primer extension sequencing can be performed after PCR amplification of regions of interest. Software can assist with sequence base calling and with mutation identification. SEQUENOM™ MASSARRAY® sequencing analysis (San Diego, Calif.) is a mass-spectrometry method which compares actual mass to expected mass of particular fragments of interest to identify mutations. NGS technology (also called “massively parallel sequencing” and “second generation sequencing”) in general provides for much higher throughput than previous methods and uses a variety of approaches (reviewed in Zhang et al. (2011) J Genet. Genomics 38:95-109 and Shendure and Hanlee (2008) Nature Biotech. 26:1135-1145). NGS methods can identify low frequency mutations in a marker in a sample. Some NGS methods (see, e.g., GS-FLX Genome Sequencer (Roche Applied Science, Branford, Conn.), Genome analyzer (Illumina, Inc. San Diego, Calif.) SOLID™ analyzer (Applied Biosystems, Carlsbad, Calif.), Polonator G.007 (Dover Systems, Salem, Nebr.), HELISCOPE™ (Helicos Biosciences Corp., Cambridge, Mass.)) use cyclic array sequencing, with or without clonal amplification of PCR products spatially separated in a flow cell and various schemes to detect the labeled modified nucleotide that is incorporated by the sequencing enzyme (e.g., polymerase or ligase). In one NGS method, primer pairs can be used in PCR reactions to amplify regions of interest. Amplified regions can be ligated into a concatenated product. Clonal libraries are generated in the flow cell from the PCR or ligated products and further amplified (“bridge” or “cluster” PCR) for single-end sequencing as the polymerase adds a labeled, reversibly terminated base that is imaged in one of four channels, depending on the identity of the labeled base and then removed for the next cycle. Software can aid in the comparison to genomic sequences to identify mutations. Another NGS method is exome sequencing, which focuses on sequencing exons of all genes in the genome. As with other NGS methods, exons can be enriched by capture methods or amplification methods.

In some embodiments, DNA, e.g., genomic DNA corresponding to the wild type or mutated marker can be analyzed both by in situ and by in vitro formats in a biological sample using methods known in the art. DNA can be directly isolated from the sample or isolated after isolating another cellular component, e.g., RNA or protein. Kits are available for DNA isolation, e.g., QIAAMP® DNA Micro Kit (Qiagen, Valencia, Calif.). DNA also can be amplified using such kits.

In another embodiment, mRNA corresponding to the marker can be analyzed both by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). RNA can be isolated using standard procedures (see e.g., Chomczynski and Sacchi (1987) Anal. Biochem.162:156-159), solutions (e.g., trizol, TM REAGENT® (Molecular Research Center, Inc., Cincinnati, Ohio; see U.S. Pat. No. 5,346,994) or kits (e.g., a QIAGEN® Group RNEASY® isolation kit (Valencia, Calif.) or LEUKOLOCK™ Total RNA Isolation System, Ambion division of Applied Biosystems, Austin, Tex.).

Additional steps may be employed to remove DNA from RNA samples. Cell lysis can be accomplished with a nonionic detergent, followed by microcentrifugation to remove the nuclei and hence the bulk of the cellular DNA. DNA subsequently can be isolated from the nuclei for DNA analysis. In one embodiment, RNA is extracted from cells of the various types of interest using guanidinium thiocyanate lysis followed by CsCl centrifugation to separate the RNA from DNA (Chirgwin et al. (1979) Biochemistry 18:5294-99). Poly(A)+RNA is selected by selection with oligo-dT cellulose (see Sambrook et al. (1989) Molecular Cloning—A Laboratory Manual (2nd ed.), Cold Spring Harbor Laboratory, Cold Spring Harbor, N.Y.). Alternatively, separation of RNA from DNA can be accomplished by organic extraction, for example, with hot phenol or phenol/chloroform/isoamyl alcohol. If desired, RNAse inhibitors may be added to the lysis buffer. Likewise, for certain cell types, it may be desirable to add a protein denaturation/digestion step to the protocol. For many applications, it is desirable to enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX® medium (see Ausubel et al. (1994) Current Protocols In Molecular Biology, vol. 2, Current Protocols Publishing, New York). Once bound, poly(A)+mRNA is eluted from the affinity column using 2 mM EDTA/0.1% SDS.

A characteristic of a marker in a sample, e.g., after obtaining a sample (e.g., a a tumor biopsy) from a test subject, can be assessed by any of a wide variety of well known methods for detecting or measuring the characteristic, e.g., of a marker or plurality of markers, e.g., of a nucleic acid (e.g., RNA, mRNA, genomic DNA, or cDNA) and/or translated protein. Non-limiting examples of such methods include immunological methods for detection of secreted, cell-surface, cytoplasmic, or nuclear proteins, protein purification methods, protein function or activity assays, nucleic acid hybridization methods, optionally including “mismatch cleavage” steps (Myers, et al. (1985) Science 230:1242) to digest mismatched, i.e. mutant or variant, regions and separation and identification of the mutant or variant from the resulting digested fragments, nucleic acid reverse transcription methods, and nucleic acid amplification methods and analysis of amplified products. These methods include gene array/chip technology, RT-PCR, TAQMAN® gene expression assays (Applied Biosystems, Foster City, Calif.), e.g., under GLP approved laboratory conditions, in situ hybridization, immunohistochemistry, immunoblotting, FISH (flourescence in situ hybridization), FACS analyses, northern blot, southern blot, INFINIUM® DNA analysis Bead Chips (Illumina, Inc., San Diego, Calif.), quantitative PCR, bacterial artificial chromosome arrays, single nucleotide polymorphism (SNP) arrays (Affymetrix, Santa Clara, Calif.) or cytogenetic analyses.

Examples of techniques for detecting differences of at least one nucleotide between two nucleic acids include, but are not limited to, selective oligonucleotide hybridization, selective amplification, or selective primer extension. For example, oligonucleotide probes can be prepared in which the known polymorphic nucleotide is placed centrally (allele- or mutant-specific probes) and then hybridized to target DNA under conditions which permit hybridization only if a perfect match is found (Saiki et al. (1986) Nature 324:163); Saiki et al (1989) Proc. Natl. Acad. Sci USA 86:6230; and Wallace et al. (1979) Nucl. Acids Res. 6:3543). Such allele specific oligonucleotide hybridization techniques can be used for the simultaneous detection of several nucleotide changes in different polymorphic or mutated regions of N-Ras. For example, oligonucleotides having nucleotide sequences of specific allelic variants or mutants are attached to a solid support, e.g., a hybridizing membrane and this support, e.g., membrane, is then hybridized with labeled sample nucleic acid. Analysis of the hybridization signal thus can reveal the identity of the nucleotides of the sample nucleic acid.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices and materials are herein described. All publications mentioned herein are hereby incorporated by reference in their entirety for the purpose of describing and disclosing the materials and methodologies that are reported in the publication which might be used in connection with the disclosure.

### EXAMPLES

Definitions

| | |
|---|---|
| ANOVA | Analysis of variance |
| ΔAUC | difference in the area under the curve |
| BID | twice daily |
| BIW | twice weekly |
| BWL | body weight loss |
| HPBCD | 2-hydroxypropyl-β-cyclodextrin |
| HPMC | hydroxypropylmethylcellulose |
| IV | intravenous(ly) |
| MTD | maximum tolerated dose |
| N/A | not applicable |
| NaHCO₃ | Sodium bicarbonate |
| SEM | Standard error of the mean |
| SCID | severe combined immunodeficiency |
| PEG | Polyethylene glycol |
| po. | Orally (by mouth, per os) |
| QD | once daily |
| QW or Q7D | once weekly |
| SC | subcutaneous(ly) |
| TG | treatment group |
| TGI | tumor growth inhibition |
| WFI | water for injection |

### Example 1

### Kinase Inhibition Assay with Purified Raf Kinase Isoforms

The kinase activity of Compound A was determined using a biochemical fluorescence resonance energy transfer (FRET) assay as described in WO 2009/006389. The half maximal inhibitory concentration (IC50) values of Compound A for mutant B-Raf V600E, wild-type B-Raf, and wild-type C-Raf kinases is shown below in Table 1. Compound A binds to the inactive, DFG-out conformation of B-Raf kinase.

TABLE 1
Biochemical kinase assay

| Raf | IC₅₀ value (nM) |
|---|---|
| B-Raf mutant (V600E) | 7.1 |
| B-Raf wild-type | 10.1 |
| C-Raf wild-type | 0.7 |

### Example 2

### In Vivo Tumor Efficacy in N-Ras Mutated SK-MEL-2 Human Melanoma Xenograft Model

### Test Compounds:

Compound A was formulated in PEG 400 and the suspension was sonicated in a warm water bath until a clear solution was obtained. The 10 mg/mL solution was diluted with 100% PEG 400 for the lower dose.

Docetaxel [Taxotere® (docetaxel) Injection Concentrate; 20 mg/mL in Ethanol/Tween 80] was diluted with saline to 1.5 mg/mL. Only docetaxel was used in non-clinical studies because of formulation limitations in mice.

The 2 vehicles, 100% PEG 400 (Vehicle 1) and 10% HPBCD/1% NaHCO₃ in WFI (Vehicle 2) were administered (0.05 mL/10 g BW) concomitantly to mice in the vehicle group.

### Tumor Measurements:

Tumor size and body weight were measured BIW beginning on the first day of treatment. Animals were terminated when their tumor reached approximately 2000 mm³, and the study was terminated on Day 62 post treatment initiation.

Inhibition of tumor growth was determined by calculating the percent TGI (MTV of the vehicle group—MTV of a treated group)/MTV of the vehicle group on Day 20 post treatment initiation.

Statistical comparisons of tumor growth between treatment groups and vehicle were conducted using a linear mixed effects regression analysis on the AAUC.

### Statistical Analysis

The differences in the tumor growth trends over time between the vehicle control and treatment groups were assessed using linear mixed effects regression models. These models take into account that each animal was measured at multiple time points. A model was fit for the comparison, and the areas under the tumor volume-versus-time curve (AUCs) for control and treatment groups were calculated using the values predicted from the model. A statistically significant p value suggests that the trends over time for the two groups (vehicle and treatment) were different.

All tumor volumes had a value of 1 added to them before log₁₀ transformation. These values were compared across treatment groups to assess whether the differences in trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ+treatᵢ+dayⱼ+dayⱼ²+(treat*day)ᵢⱼ+(treat*day²)ᵢⱼ+eᵢⱼₖ (1)

where Yᵢⱼₖ is the log₁₀ tumor value at the j^{th} time point of the k^{th} animal in the i^{th} treatment, Yᵢ₀ₖ is the day 0 (baseline) log₁₀ tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if there were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model.

The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} \times 100 \qquad ( 2 )$$

This assumed AUC_{ctl} was positive. In instances where AUC_{ctl} was negative, the above formula was multiplied by −1.

For synergy analysis, the observed differences in the log tumor values were used to calculate AUC values for each animal. In instances when an animal in a treatment group was removed from the study, the last observed tumor value was carried forward through all subsequent time points. The AUC for the control, or vehicle group was calculated using the predicted values from pairwise models described above. A measure of synergy was defined as follows:

$$\begin{matrix} {Frac}_{{A}_{k}} = \frac{{AUC}_{ctl} - {AUC}_{{A}_{k}}}{{AUC}_{ctl}} & ( 3 ) \\ {Frac}_{{B}_{k}} = \frac{{AUC}_{ctl} - {AUC}_{{B}_{k}}}{{AUC}_{ctl}} & ( 4 ) \\ {Frac}_{{AB}_{k}} = \frac{{AUC}_{ctl} - {AUC}_{{AB}_{k}}}{{AUC}_{ctl}} & ( 5 ) \\ synergy score = ( mean ( {Frac}_{A} ) + mean ( {Frac}_{B} ) - mean ( {Frac}_{AB} ) ) * 100 & ( 6 )\end{matrix}$$

where Aₖ and Bₖ are the k^{th} animal in the individual treatment groups and ABₖ is the k^{th} animal in the combination treatment group. AUC_{ctl} is the model-predicted AUC for the control group and was treated as a constant with no variability. The standard error of the synergy score was calculated as the square root of the sum of the squared standard errors across groups A, B, and AB. The degrees of freedom were estimated using the Welch-Satterthwaite equation. A hypothesis test was performed to determine if the synergy score differed from 0. P values were calculated by dividing the synergy score by its standard error and tested against a t-distribution (two-tailed) with the above-calculated degrees of freedom.

The effect was classified into four different categories. It was considered synergistic if the synergy score was less than 0 and additive if the synergy score wasn't statistically different from 0. If the synergy score was greater than zero, but the mean AUC for the combination was lower than the lowest mean AUC among the two single agent treatments, then the combination was sub-additive. If the synergy score was greater than zero, and the mean AUC for the combination was greater than the mean AUC for at least one of the single agent treatments, then the combination was antagonistic.

Interval analysis, if requested, involved a specified treatment group and time interval compared with another treatment group and time interval. For a given group, time interval, and animal, the tumor growth rate per day was estimated by

Rate=100*(10^{ΔY/Δt}−1) (7)

where ΔY is the difference in the log10 tumor volume over the interval of interest, and Δt is the length of the time interval. If one or both of the time points were missing, then the animal was ignored. The mean rates across the animals were then compared to using a two-sided unpaired t-test with unequal variances. There were no adjustments pre-specified for the multiple comparisons and endpoints examined. All P values<0.05 were called statistically significant. Synergistic analysis: p>0.05=additive; p<0.05 and score<0=synergistic; p<0.05, score>0, and the combination growth rate is lower than both the single agent growth rates=subadditive; p<0.05, score>0, and the combination growth rate is higher than at least one of the single agent growth rates=antagonistic.

### Results

Combination treatment with Compound A (12.5 mg/kg QD or 50 mg/kg BIW) and docetaxel (IV, 7.5 mg/kg QW; TGI=92.7% or 91.3%, respectively) resulted in a therapeutic advantage over single agent therapy and the interaction of the two agents was additive. In the combination group treated with Compound A at 12.5 mg/kg QD and docetaxel at 7.5 mg/kg Q, there were two complete tumor regressions with one animal remaining tumor-free at study termination. The detail for this study is shown below in Table 2.

TABLE 2
Summary of Results (N-Ras mutated SK-MEL-2 human melanoma)

| | | Method of | Sex/No. | | | |
|---|---|---|---|---|---|---|
| Treatment | Dose | Administration/ | Per | Species/ | | |
| Group | (mg/kg) | Frequency | Group | Strain | Endpoints | Findings |
| Vehicle 1 + | 0.0 | PO/QD | F/8 | Mouse | TGI^{a} | N/A |
| Vehicle 2 | | Days 0-20 | | (Mus musculus) | Maximum | 0% |
| | | | | Athymic NCr- | Mean | |
| | | | | nu/nu | % BWL^{b} | |
| Compound A + | 12.5 | PO/QD | F/8 | Mouse | TGI | 70.7% |
| Vehicle 2 | | Days 0-20 | | (Mus musculus/ | ΔAUC | p < 0.001 |
| | | | | Athymic NCr- | Mean Maximum | 1% (Days |
| | | | | nu/nu | BWL ^{b} | 3 and 10) |
| Compound A + | 50 | PO/BIW | F/8 | Mouse | TGI | 62.9% |
| Vehicle 2 | | Days 0, 3, | | (Mus musculus/ | ΔAUC | p < 0.001 |
| | | 7, 10, | | Athymic NCr- | Mean Maximum | 4% |
| | | 14, and 17 | | nu/nu | BWL ^{b} | (Day 17) |
| | | | | | ΔAUC | p < 0.001 |
| | | | | | Mean Maximum | 4% |
| | | | | | BWL ^{b} | (Day 14) |
| docetaxel + | 7.5 | IV/QW | F/8 | Mouse | TGI | 74.9% |
| Vehicle 1 | | Days 0, | | (Mus musculus/ | ΔAUC | p < 0.001 |
| | | 7, and 14 | | Athymic NCr- | Mean Maximum | 15% |
| | | | | nu/nu | BWL ^{b} | (Day 14) |
| | | | | | ΔAUC^{c} | p < 0.001 |
| | | | | | Synergy analysis^{d} | Additive |
| | | | | | | p = 0.524 |
| | | | | | Mean Maximum | 7% |
| | | | | | BWL^{b} | (Day 14) |
| | | | | | Synergy analysis^{d} | Subadditive |
| | | | | | | p = 0.042 |
| | | | | | Mean Maximum | 9% |
| | | | | | BWL^{b} | (Day 14) |
| | | | | | ΔAUC^{c} | p < 0.001 |
| | | | | | Synergy analysis^{d} | Additive |
| | | | | | | p = 0.404 |
| | | | | | Mean Maximum | 5% |
| | | | | | BWL^{b} | (Day 14) |
| Compound A + | 12.5 | PO/QD | F/8 | Mouse | TGI | 92.7% |
| docetaxel | 7.5 | Days 0-20 | | (Mus musculus/ | ΔAUC^{c} | p < 0.001 |
| | | IV/QW | | Athymic NCr- | Synergy analysis^{d} | Additive |
| | | Days 0, | | nu/nu | | p = 0.344 |
| | | 7, and 14 | | | Mean Maximum | 10% |
| | | | | | BWL^{b} | (Day 14) |
| Compound A + | 50 | PO/BIW | F/8 | Mouse | TGI | 91.3% |
| docetaxel | 7.5 | Days 0, 3, | | (Mus musculus/ | ΔAUC^{c} | p < 0.001 |
| | | 7, 10, | | Athymic NCr- | Synergy | Additive |
| | | 14, and 17 | | nu/nu | analysis^{d} | p = 0.252 |
| | | IV/QW | | | Mean | 16% |
| | | Days 0, | | | Maximum | (Day 17) |
| | | 7, and 14 | | | BWL^{b} | |

^{a}Dose volume for each vehicle was 0.05 mL/10g BW
^{b} TGI values were calculated on Day 20 post treatment initiation.
^{c}Maximum mean percent BWL.
^{d}ΔAUC = Statistical analysis was performed with a linear mixed effects regression model. A p value of <0.05 was considered significant.
^{e} Calculated on Day 20 post treatment initiation.

### Example 3

### Methods for Measuring Markers

B-Raf PCR based Assay (Vendor: Qiagen; Catalog #: 870801)

The B-Raf RGQ PCR Kit v2 combines two technologies, ARMS® and Scorpions®, to detect mutations in real-time PCR assays. This assay detects B-Raf V600 mutations V600E (GAG) and V600E complex (GAA), V600D (GAT), V600K (AAG), V600R (AGG). The kit detects the presence of the V600E (GAG) and V600E complex (GAA) but does not distinguish between them.

### ARMS

Specific mutated sequences are selectively amplified by allele specific primer designed to match a mutated DNA.

### Scorpions

Detection of amplification is performed using Scorpions. Scorpions are PCR primer covalently linked to a fluorescently labeled probe (i.e. FAM™ or HEX™) and a quencher. During PCR when the probe is bound to the amplicon, the fluorophore and quencher become separated resulting in an increase in fluorescence signal.

### Procedure

The B-Raf RGQ PCR Kit v2 comprises a two-step procedure. In the first step, the control assay is performed to assess the total amplifiable B-Raf DNA in a sample. In the second step, both the mutation and control assays are performed to determine the presence or absence of mutant DNA.

### Control Assay

The control assay, labeled with FAM, is used to assess the total amplifiable B-Raf DNA in a sample. The control assay amplifies a region of exon 3 of the B-Raf gene. The primers and Scorpion probe are designed to amplify independently of any known B-Raf polymorphisms.

### Mutation Assays

Each mutation assay contains a FAM-labeled Scorpion probe and an ARMS primer for discrimination between the wild-type DNA and a specific mutant DNA.

### Data Analysis: ΔCt Method

Scorpions real-time assays uses the number of PCR cycles necessary to detect a fluorescent signal above a background signal as a measure of the target molecules present at the beginning of the reaction. The point at which the signal is detected above background fluorescence is called the ‘cycle threshold’ (Ct).

Sample ΔCt values are calculated as the difference between the mutation assay Ct and control assay Ct from the same sample. Samples are classed as mutation positive if they give a ΔCt less than the Cut-Off ΔCt value for that assay. Above this value, the sample either contains less than the percentage of mutation able to be detected by the kit (beyond the limit of the assays), or the sample is mutation negative.

When using ARMS primers some inefficient priming could occur, giving a very late background Ct from DNA not containing a mutation. All ΔCt values calculated from background amplification are, greater than the cut off ΔCt values and the sample is classed mutation negative.

For each sample, the ΔCt values are calculated as follows, ensuring that the mutation and control Ct values are from the same sample:

ΔCt={sample mutation Ct}−{sample control Ct}
Sample control Ct can range between 27-33
Sample mutation Ct can range between 15-40

Acceptable ΔCt for the mutant call is <6 or 7

Methods for measuring N-Ras mutations are similar to those described above for B-Raf. Qiagen N-Ras assay for the detection of N-Ras Q61 mutations includes:
Q61K (181 C>A)
Q61R (182 A>G)

Methods for measuring K-Ras mutations are readily available. For example, Qiagen TheraScreen® K-Ras mutation kit.

### Example 4

### Compound A and Docetaxel Combination in KRAS Mutant Lung Cancer Models

Compound A and docetaxel combination was evaluated in KRAS mutant lung cancer models including Calu-6 lung cancer model (KRAS^{Q61K}) and Δ549 lung cancer model (KRAS^{G12S}). The results of the Calu-6 lung cancer model are described below. In the A549 model, the combination showed no better activity than single agents.

For the Calu-6 model, Compound A was formulated 100% PEG400 as follows:

1. Compound A at 10.0 mg/mL: 4.0 mL
Weighed out 40.40 mg of Compound A powder;
Added the powder in 4 mL of 100% PEG400;
Sonicated until no Compound A particles remained and a solution was achieved;
Stored at room temperature until use.
   2. Compound A at 2.5 mg/mL: 20.0 mL
Weighed out 50.51 mg of Compound A powder;
Added the powder in 20 mL of 100% PEG400;
Sonicated until no drug particles remained and a solution was achieved;
Stored at room temperature until use.

Docetaxel (e.g., Taxotere Injection Concentrate, Sanofi-Aventis U.S. LLC) was formulated in 0.9% saline as follows:

1. Taxotere at 0.75 mg/mL: 8 mL
0.3 mL of Taxotere Injection Concentrate (20 mg/mL) was added into a centrifuge tube;
Added 7.7 mL normal saline and mixed manually;
Kept the tube protected from light and used within 4 hours.
   2. Taxotere at 0.50 mg/mL: 6 mL
0.15 mL of Taxotere Injection Concentrate (20 mg/mL) was added into a centrifuge tube;
Added 5.85 mL normal saline and mixed manually;
Kept the tube protected from light and used within 4 hours.

Taxotere Injection Concentrate, instead of docetaxel, was used in recording raw data.

Animals in the vehicle treatment group were given 100% PEG400. The dose volume for vehicle or compound was 5 mL/kg body weight for PO and 10 mL/kg for IV administration.

The animals used in this study were Balb/c nude female mice (Shaghai SINO-British SIPPR/BK Lab Animal Ltd). The number of animals per group was 7. The age of the mice at Day 0 was 9 weeks. Group average weight at day 0 was 20.1-21.5 g.

Table 3 shows the dosing regimens for each treatment group used in the study.

TABLE 3
Summary of Results (KRAS mutated Calu-6 human lung carcinoma).

| | | Method of | Sex | | | |
|---|---|---|---|---|---|---|
| Treatment | Dose | Administration/ | Number | Species/ | | |
| Group | (mg/kg) | Frequency | Per Group | Strain | Endpoints | Findings |
| Vehicle ^{a} | 0.0 | PO/QD | F/7 | Mouse | TGI ^{b} | N/A |
| | | Days 1-21 | | (Mus musculus) | Mean | 0% |
| | | | | Athymic | Maximum | |
| | | | | Balb/c Nude | % BWL^{c} | |
| Compound A | 12.5 | PO/QD | F/7 | Mouse | TGI ^{b} | 58.9% |
| | | Days 1-21 | | (Mus musculus) | ΔAUC ^{d} | 55.4 (p < 0.001) |
| | | | | Athymic | Mean Maximum | 0% |
| | | | | Balb/c Nude | % BWL ^{c} | |
| Compound A | 50 | PO/BIW | F/7 | Mouse | TGI ^{b} | 59.4% |
| | | Days 1,4, | | (Mus musculus) | ΔAUC ^{d} | 62.7 (p < 0.001) |
| | | 8, 11 | | Athymic | Mean Maximum | −0.1% |
| | | 15, and 18 | | Balb/c Nude | % BWL ^{c} | (Day 7) |
| | | | | | ΔAUC ^{d} | 29.7 (p < 0.01) |
| | | | | | Mean Maximum | 0% |
| | | | | | % BWL ^{c} | |
| docetaxel | 5 | IV/QW | F/7 | Mouse | TGI ^{b} | 5.2% |
| | | Days 1, | | (Mus musculus) | ΔAUC ^{d} | 9.0 (p > 0.05) |
| | | 8, and 15 | | Athymic | Mean Maximum | 0% |
| | | | | Balb/c Nude | % BWL ^{c} | |
| docetaxel | 7.5 | IV/QW | F/7 | Mouse | TGI ^{b} | 17.7% |
| | | Days 1, | | (Mus musculus) | ΔAUC ^{d} | 18.4 (p < 0.05) |
| | | 8, and 15 | | Athymic | Mean Maximum | 2.5% |
| | | | | Balb/c Nude | % BWL^{c} | (Day 21) |
| Compound A + | 12.5 | PO/QD | F/7 | Mouse | TGI ^{b} | 69.1% |
| docetaxel | 5 | Days 1-21 | | (Mus musculus) | ΔAUC ^{d} | 71.3 (p < 0.001) |
| | | IV/QW | | Athymic | Synergy analysis ^{e} | Additive (p = 0.614) |
| | | Days 1, | | Balb/c | Mean Maximum | 8.9% |
| | | 8, and 15 | | Nude | % BWL ^{c} | (Day 21) |
| Compound A + | 12.5 | PO/QD | F/7 | Mouse | TGI ^{b} | 69.4% |
| docetaxel | 7.5 | Days 1-21 | | (Mus musculus) | ΔAUC ^{d} | 75.4 (p < 0.001) |
| | | IV/QW | | Athymic | Synergy analysis ^{e} | Additive (p = 0.926) |
| | | Days 1, | | Balb/c Nude | Mean Maximum | 14.0% |
| | | 8, and 15 | | | % BWL^{c} | (Day 21) |
| Compound A + | 50 | PO/BIW | F/7 | Mouse | TGI ^{b} | 63.9% |
| docetaxel | 7.5 | Days 1, 4, | | (Mus musculus) | ΔAUC ^{d} | 71.5 (p < 0.001) |
| | | 8, 11, | | Athymic | Synergy | Additive |
| | | 15, and 18 | | Balb/c Nude | analysis ^{e} | (p = 0.360) |
| | | IV/QW | | | Mean | 8.0% |
| | | Days 1, | | | Maximum | (Day 21) |
| | | 8, and 15 | | | BWL^{c} | |

ΔAUC = change in areas under the tumor volume-versus-time curves; BIW = twice weekly; BWL = body weight loss; IV = intravenously; N/A = not applicable; QD = daily; QW = weekly; TGI = tumor growth inhibition; Vehicle =100% PEG400.
^{a} Dose volume for vehicle or compound was 5 mL/kg body weight for PO and 10 mL/kg for IV administration.
^{b} TGI values were calculated on Day 21 post treatment initiation.
^{c} Maximum mean percent BWL between Day 0 to Day 21.
^{d} ΔAUC = Statistical analysis was performed with a linear mixed effects regression model. A p value of <0.05 was considered significant.
^{e} Calculated on Day 21 post treatment initiation.

Each animal was inoculated with 2×10⁶ Calu-6 tumor cells (in 0.1 mL, 1:1 with Matrigel) at the right flank for tumor model development. Body weight and the tumor growth were monitored twice weekly. Tumor size was measured to the nearest 0.1 mm using vernier caliper and applying the formula V=W²×L/2, where V=volume, W=width, and L=length for the tumor xenograft. Xenografts were allowed to grow until they reached an average size of approximately 220 mm³ after 14 days. Mice bearing the proper size xenograft were randomly assigned into one of the groups shown in the table above and began to be treated with their assigned test materials, either vehicle (100% PEG400), Compound A (at 12.5 or 50 mg/kg), docetaxel (at 5 or 7.5 mg/kg), or the combination of Compound A/docetaxel.

Tumor size and body weight were measured twice weekly beginning on the day of animal grouping. The study was terminated on Day 35 post treatment initiation.

Inhibition of tumor growth was determined by calculating the percent TGI (MTV of the vehicle group—MTV of a treated group)/MTV of the vehicle group] on Day 21 of the study. Statistical comparisons of tumor growth between treatment groups and vehicle were conducted using a linear mixed effects regression analysis on the AAUC.

Statistical Tests

Delta AUC

The differences in the tumor growth trends over time between the vehicle control and treatment groups were assessed using linear mixed effects regression models. These models take into account that each animal was measured at multiple time points. A model was fit for the comparison, and the areas under the tumor volume-versus-time curve (AUCs) for control and treatment groups were calculated using the values predicted from the model. A statistically significant p value suggests that the trends over time for the 2 groups (vehicle and treatment) were different. A p value <0.05 was considered statistically significant. Further details regarding pair-wise comparisons are provided Example 2.

A combination score calculation was used to address the question of whether the effects of the combination treatments were synergistic, additive, subadditive, or antagonistic relative to the individual treatments. The effect was considered synergistic if the synergy score was less than 0, and additive if the synergy score wasn't statistically different from 0. If the synergy score was greater than 0, but the mean AUC for the combination was lower than the lowest mean AUC among the two single agent treatments, then the combination was subadditive. If the synergy score was greater than zero, and the mean AUC for the combination was greater than the mean AUC for at least one of the single agent treatments, then the combination was antagonistic.

What is claimed:

1. A method of treating a subject suffering from cancer, comprising administering to the subject:
(iii) a Raf kinase inhibitor or a pharmaceutically acceptable salt thereof; and
(iv) a taxane or a pharmaceutically acceptable salt thereof; the amount of Raf kinase inhibitor and taxane or a pharmaceutically acceptable salt thereof is such that the combination thereof is therapeutically effective in the treatment of the cancer.

2. The method claim 1, wherein the Raf kinase inhibitor inhibits more than the B-Raf V600 isoform of Raf proteins.

3. The method of any one of claims 1-2, wherein the Raf inhibitor is selected from

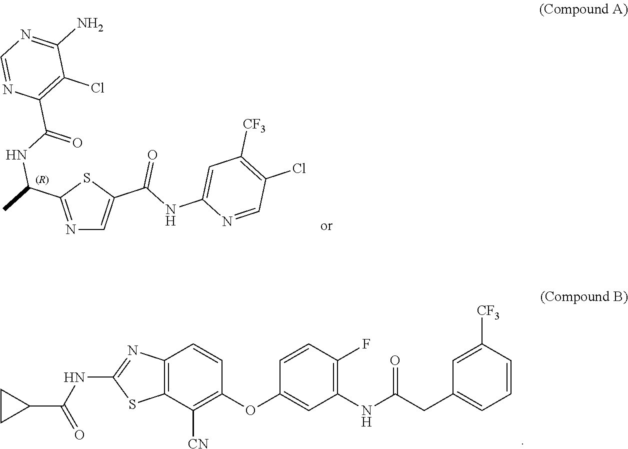

4. The method of any one of claims 1-3, wherein the taxane is paclitaxel, docetaxel (taxotere), or Abraxane® or a pharmaceutically acceptable salt thereof.

5. The method of claim 4, wherein the taxane is paclitaxel or a pharmaceutically acceptable salt thereof.

6. The method of any one of claims 1-5, wherein the cancer is a K-Ras exon 2 mutation positive cancer.

7. The method of any one of claims 1-6, wherein the cancer is a B-Raf mutation positive cancer.

8. The method of claim 7, wherein the cancer is a non-V600 B-Raf mutation positive cancer.

9. The method of any one of claims 1-8, wherein the cancer is selected from skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, endometrial cancer, uterine cancer, liver cancer, lung cancer, prostate cancer, and colon cancer.

10. A method of treating a subject suffering from cancer, comprising administering to the subject:
(iii) Compound A: 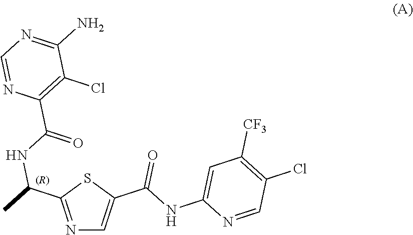
or a pharmaceutically acceptable salt thereof; and
(iv) paclitaxel or docetaxel or a pharmaceutically acceptable salt thereof; the amount of said active agents is such that the combination thereof is therapeutically effective in the treatment of the cancer.

11. The method of claim 10, wherein the cancer is non-small cell lung cancer.

12. The method of claim 11, wherein the non-small cell lung cancer is a K-Ras mutation positive cancer.

13. The method of claim 11, wherein the non-small cell lung cancer is a B-Raf mutation positive cancer.

14. The method of any one of claims 10-13, wherein Compound A or a pharmaceutically acceptable salt thereof, is administered administered once weekly (QW) with a rest period of 6 days between each administration in an amount of up to 600 mg per dose.

15. The method of claim 14, wherein Compound A or a pharmaceutically acceptable salt thereof, is administered in an amount of about 400 mg to about 600 mg per dose.

16. The method of any one of claims 10-15, wherein the taxane is paclitaxel or a pharmaceutically acceptable salt thereof administered in an amount of from about 70 mg/m² to about 90 mg/m² per dose.

17. A pharmaceutical composition comprising:
(iii) a Raf kinase inhibitor or a pharmaceutically acceptable salt thereof; and
(iv) a taxane or a pharmaceutically acceptable salt thereof; the amount of said Raf kinase inhibitor and taxane or a pharmaceutically acceptable salt thereof is such that the combination thereof is effective for extending the duration of response.

18. The composition of claim 17, wherein the Raf kinase inhibitor is Compound A or Compound B.
